(19) United States
(12) Patent Application Publication
(10) Pub. No.: US 2013/0310448 A1
(43) Pub. Date: Nov. 21, 2013

(54) METHODS AND COMPOSITIONS FOR INHIBITION OF ATR AND FANCD2 ACTIVATION

(71) Applicant: KAOHSIUNG MEDICAL UNIVERSITY, Kaohsiung (TW)

(72) Inventors: Hui-Chun WANG, Kaohsiung (TW); Yang-Chang WU, Kaohsiung (TW); Fang-Rong CHANG, Kaohsiung (TW); Chin-Chung WU, Kaohsiung (TW)

(73) Assignee: KAOHSIUNG MEDICAL UNIVERSITY, Kaohsiung (TW)

(21) Appl. No.: 13/714,109

(22) Filed: Dec. 13, 2012

(30) Foreign Application Priority Data
May 18, 2012 (TW) 101117920

(51) Int. Cl. A61K 31/352; C07D 309/30; A61K 45/06

(52) U.S. Cl. CPC A61K 31/352; A61K 45/06; C07D 309/30
USPC 514/455; 514/456; 435/15

(57) ABSTRACT

This invention is announcing a composition of flavonoid skeleton in the formula I or formula II compound, wherein each of the substituents is given the definition as set forth in the specification and claims. This composition have the capacity to Inhibit functions of ATR and FANCD2 on DNA replication, damage checkpoint, and repair; therefore, this composition can improve the cancer sensitivity and poor prognosis to DNA-damaging therapeutics.

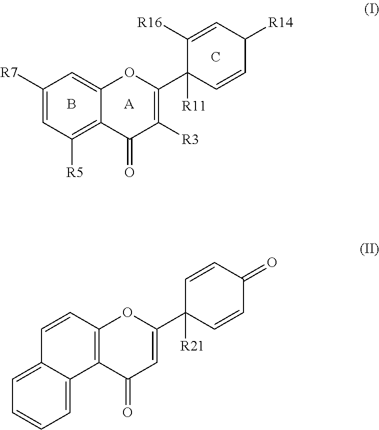

### CROSS REFERENCE TO RELATED APPLICATIONS

This application claims benefit under 35 U.S.C. §119 of Taiwanese Patent Application No. 101117920, filed on May 18, 2012, the contents of which are incorporated by reference as if fully set forth herein in its entirety.

### FIELD OF THE INVENTION

The present invention relates to benzopyran-4-one derivatives compound capable of inhibiting ATR and FANCD2 activation, and more particularly to those benzopyran-4-one derivatives compound capable of improving the cancer sensitivity and poor prognosis to DNA-damaging therapeutics.

### BACKGROUND OF THE INVENTION

The benzopyran-4-one derivatives compound of formula I having a flavonoid moiety, which previously isolated and identified from the whole plant extract of Thelypteris torresiana, a fern species native to Taiwan.

The exposure and biological effects of Protoapigenone (I-1), 5′,6′-Dihydro-6′-methoxy-protoapigenone (I-2) and Protoapigenin (I-3) compounds have been investigated by cytotoxicity assay. It was found Protoapigenone (I-1) demonstrated therapeutic effects and was a lead compound for potential anticancer drug development.

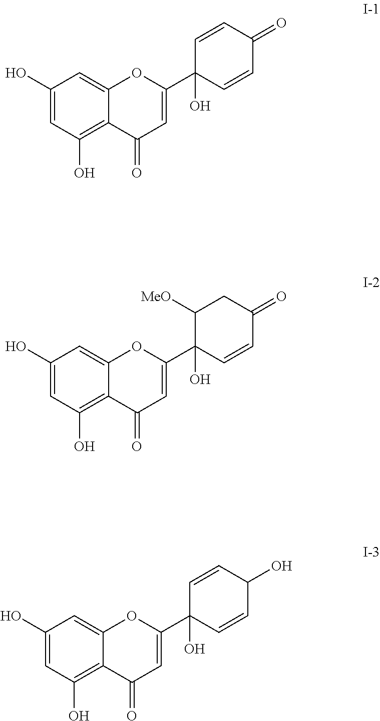 [SMILES: O=C1C=CC(O)(c2cc(=O)c3c(O)cc(O)cc3o2)C=C1 ; COC1CC(=O)C=CC1(O)c1cc(=O)c2c(O)cc(O)cc2o1 ; O=c1cc(C2(O)C=CC(O)C=C2)oc2cc(O)cc(O)c12]

Furthermore, for developing a new potent anticancer drug, several analogues of I type compound such as I-4, I-5, I-6, I-7 and I-8, and another II type moiety such as II-1 and II-2 compound are also synthesized or semi-synthesized. Where the compound I-4 having chemical name 2-(1-hydroxy-4-oxocyclohexa-2,5-dienyl)-4H-chromen-4-one can be expressed by the general names of protoflavonone. The compound I-5 is also termed as 5-hydroxyprotoflavone, whose chemical name is 2-(1-hydroxy-4-oxocyclohexa-2,5-dienyl)-5-hydroxy-4H-chromen-4-one. The compound I-6 having chemical name 5-hydroxy-2-(1-hydroxy-4-oxocyclohexa-2,5-dienyl)-7-methoxy-4H-chromen-4-one can be expressed by the general names of 5-hydroxy-7-methoxy-protoflavonone. The homologous compounds I-4 and 1-7 have the similar structure, but a different function group on R11 positions only, which is a hydroxyl group and another is methoxyl group in that position. The compound I-5, I-8, II-1 and II-2 also present the modified function group of R11 positions models.

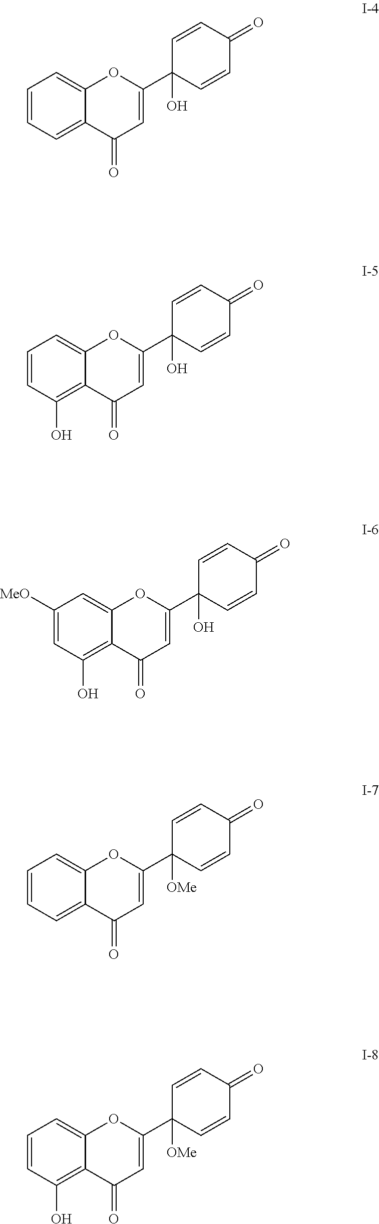 [SMILES: O=C1C=CC(O)(c2cc(=O)c3ccccc3o2)C=C1 ; O=C1C=CC(O)(c2cc(=O)c3c(O)cccc3o2)C=C1 ; COc1cc(O)c2c(=O)cc(C3(O)C=CC(=O)C=C3)oc2c1 ; COC1(c2cc(=O)c3ccccc3o2)C=CC(=O)C=C1 ; COC1(c2cc(=O)c3c(O)cccc3o2)C=CC(=O)C=C1]

Formula II having an β-naphthoflavone moiety, the compound II-1 has chemical name 3-(1-hydroxy-4-oxo-cyclohexa-2,5-dienyl)-1H-benzo[f]chromen-1-one. The compound II-2 has chemical name 1′-methoxy-β-naphthoflavone.

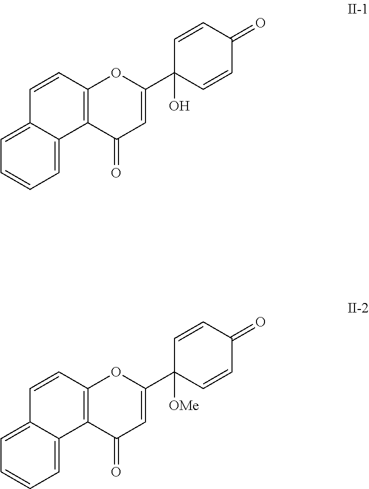 [SMILES: O=C1C=CC(O)(c2cc(=O)c3c(ccc4ccccc43)o2)C=C1 ; COC1(c2cc(=O)c3c(ccc4ccccc43)o2)C=CC(=O)C=C1]

In previous studies, Protoapigenone (I-1) and its more potent analog compound II-1 (FIG. 1A) were shown to induce oxidative stress, consequently activating the p38 and JNK1/2 MAPK pathways following cell cycle arrest and apoptosis in several cancer cell types. These compounds were also found to reduce the size of tumor xenografts in nude mice without exerting toxic effects on the recipient. Recently, in those compounds of formula I and II were found to induce chromosomal breakage through oxidative stress, implicating a role for benzopyran-4-one derivatives of formula I and II in interfering with DNA metabolism. Up to date, the biomolecular actions and implications of this benzopyran-4-one derivatives mediated interference are mostly undetermined. Herein, it is found that benzopyran-4-one derivatives are capable of inhibiting DNA damage-induced activation of ATR targets Chk (Cell Cycle Checkpoint Kinase) 1 and FANCD2, which then sensitize tumor cells to chemotherapy, and finally results in tumor size reduction in mice. The experiment results show that these benzopyran-4-one derivatives compounds are noteworthy potential to treat cancers by inducing replication stress via the inhibition of ATR signaling cascades.

### SUMMARY OF THE INVENTION

In accordance with an aspect of the present invention, compounds of benzopyran-4-one derivatives, characteristically with inhibiting of DNA Damage Response (DDR), are provided. The benzopyran-4-one derivatives includes a common structure being the following formula I:

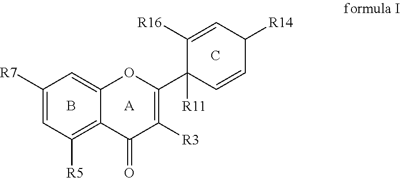

wherein: R₃, R₅, R₇, R₁₁, R₁₄ and R₁₆ are selected independently from a group consisting of a hydrogen, a hydroxyl group, a methoxyl group and a oxygen atom contain a double bond.

In accordance with a further aspect of the present invention, compounds of benzopyran-4-one derivatives, characteristically with ATR-mediated DNA damage checkpoint, is provided. The benzopyran-4-one derivatives includes a common structure being the following formula II;

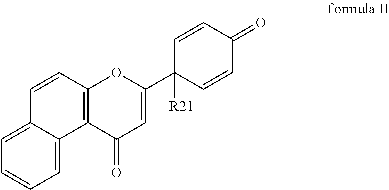

wherein: R₂₁ is selected independently from a group consisting of a hydrogen, a hydroxyl group and a methoxyl group.

In a further aspect of the present invention, a method for assaying a state of DNA DDR kinase signaling cascades is provided. The method includes steps of:
providing a reaction site thereof;
adding to the reaction site an effective amount of a benzopyran-4-one derivative represented by one of formula I and formula II,

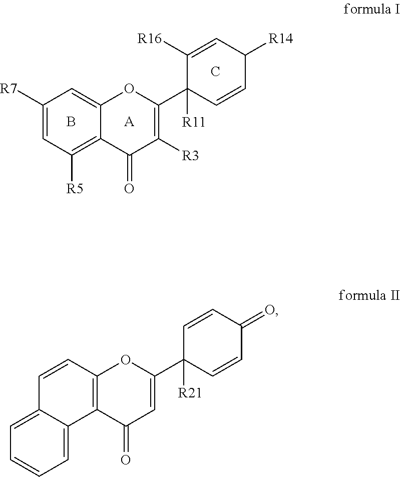

wherein each of R₃, R₅, R₇, R₁₁, R₁₄, and R₁₆ is one selected from a group consisting of a hydrogen, a hydroxyl group, a methoxyl group and a oxygen atom containing a double bond. R₂₁ is one selected from a group consisting of a hydrogen, a hydroxyl group and a methoxyl group.

The above objects and advantages of the present aspects will become more readily apparent to those ordinarily skilled in the art after reviewing the following detailed descriptions and accompanying drawings, in which:

### BRIEF DESCRIPTION OF THE DRAWINGS

The patent or application file contains at least one drawing executed in color. Copies of this patent or patent application publication with color drawing(s) will be provided by the Office upon request and payment of the necessary fee

FIG. 1 illustrates Protoapigenone (I-1) induce chromosome aberration but does not produce marked DDR.

FIG. 2 illustrates immunoblots showing DDR by detecting phosphorylation of cell marker following exposure of HEK293T cell to 10 μM compound I-1 for the indicated times.

FIGS. 3(a)-3(b) show that dose-dependent effects of compound I-1 and compound II-1 on the inhibition of UV-induced Chk1 phosphorylation in cells.

FIG. 3(a) illustrates immunoblots showing the expression of MDA-MB-231 cell.

FIG. 3(b) illustrates immunoblots showing the expression of A549 cell.

FIG. 4 shows the cytotoxic effect of compound against cell line.

FIGS. 5(a)-5(b) show benzopyran-4-one derivatives inhibit DNA damage-induced DDR.

FIG. 5(a) illustrates immunoblots showing the expression of inhibit Chk1 phosphorylation.

FIG. 5(b) illustrates immunoblots showing the expression of A549 cell. Cells pretreated with 50 μM okadaic acid (OA) or 20 μM MG132 and subjected to 10 J/m² UV for 1 h to induce DDR.

FIGS. 6(a)-6(b) show benzopyran-4-one derivatives inhibit UV-induced Chk1 phosphorylation.

FIG. 6(a) illustrates immunoblots showing the expression of A549 cell.

FIG. 6(b) illustrates immunoblots showing the expression of MDA-MB-231 cell. Cells pretreated with chemicals for 20 min and subjected to 10 J/m² UV for 1 h to induce DDR.

FIG. 7 shows benzopyran-4-one derivatives inhibit chemotherapeutic agents-induced Chk1 phosphorylation.

FIGS. 8(a)-8(b) show benzopyran-4-one derivatives inhibit ATR-dependent Chk1 phosphorylation.

FIG. 8(a) illustrates immunoblots showing the expression DDR induced by H₂O₂ (peroxide).

FIG. 8(b) illustrates immunoblots showing the expression DDR induced by hydroxyurea (HU).

FIG. 9 shows the effect of compound II-1 on phosphorylation were assayed after UV irradiation on HEK293T cell, and this exhibited decreased expression of genes following the RNA interference treatment.

FIGS. 10(a)-10(c) show benzopyran-4-one derivatives inhibit UV- or H₂O₂-induced Chk1 phosphorylation

FIG. 10(a) illustrate immunoblots showing compound I-1 inhibit H₂O₂-induced Chk1 phosphorylation.

FIG. 10(b) illustrate immunoblots showing compound I-1 inhibit UV-induced Chk1 phosphorylation.

FIG. 10(c) illustrate immunoblots showing compound II-1 inhibit H₂O₂-induced Chk1 phosphorylation.

FIG. 11 illustrate immunoblots showing compound I-1 inhibit H₂O₂-induced Chk1 phosphorylation. Effects of compound I-1 on Chk1 phosphorylation were assayed 1 h after 10 J/m² UV irradiation on HEK293T cell overexpressing ATRIP, TopBP1, claspin, or ATR following delivery of tagged full-length cDNA constructs for 48 h.

FIGS. 12(a)-12(b) show benzopyran-4-one derivatives inhibit DNA damage checkpoint and repair.

FIG. 12(a) illustrates percentages of the mitotic marker.

FIG. 12(b) illustrates the expression FACS (Fluorescence Activated Cell Sorting) dot blot for analyzing the percentage of GFP cell denoting the HRR frequency.

FIG. 13 illustrates the percentage of M-phase cells with γH2AX foucus formation.

FIGS. 14(a)-14(c) show the benzopyran-4-one derivatives inhibit cisplatin-induced Chk1 phosphorylation and FANCD2 monoubiquitination.

FIG. 14(a) illustrates immunoblots showing the effect on cisplatin-induced DDR in A594 cell.

FIG. 14(b) illustrates immunoblots showing the effect on cisplatin-induced DDR in U2OS cell.

FIG. 14(c) illustrates immunoblots showing the effect on cisplatin-induced DDR in MDA-MB-231 cell.

FIGS. 15(a)-15(b) show that in vitro clonogenic survival for A549 cell.

FIG. 15(a) illustrates the effects fraction of compound I-1.

FIG. 15(b) illustrates the effects fraction of compound II-1.

FIGS. 16(a)-16(b) show that in vitro clonogenic survival for MDA-MB-231 cell.

FIG. 16(a) illustrates the effects fraction of compound I-1.

FIG. 16(b) illustrates the effects fraction of compound II-1.

FIGS. 17(a)-17(b) show chemosensitization effect of benzopyran-4-one derivatives.

FIG. 17(a) illustrates the effects fraction of compound I-1.

FIG. 17(b) illustrates the effects fraction of compound II-1.

FIG. 18 shows that in vivo xenograft tumor volume for MDA-MB-231 cell.
A—cisplatin 2 mg/kg
B—cisplatin+compound II-1 0.2 mg/kg
C—compound II-1 0.2 mg/kg

FIGS. 19(a)-19(b) show that optical activity of benzopyran-4-one derivatives.

FIG. 19(a) illustrates the effects on A549 cells
A—control
B—0.25 μM compound I-1
C—0.5 μM compound I-1
D—1 μM compound I-1

FIG. 19(b) illustrates the effects on MDA-MB-231 cells
A—control
B—0.25 μM compound I-1
C—0.5 μM compound I-1
D—1 μM compound I-1

FIGS. 20(a)-20(b) show that optical activity affected by benzopyran-4-one derivatives concentration.

FIG. 20(a) illustrates the effects of compound I-1.
A—control
B—0.1 μM compound II-1
C—0.2 μM compound II-1
D—0.4 μM compound II-1

FIG. 20(b) illustrates the effects of compound II-1.
A—control
B—0.1 μM compound II-1
C—0.2 μM compound II-1
D—0.4 μM compound II-1

FIGS. 21(a)-21(b) show that rate of cell cycle progression.

FIG. 21(a) illustrates the effects of unsynchronized cells.

FIG. 21(b) illustrates the effects of control group.

FIGS. 22(a)-22(c) show that rate of cell cycle progression were treated with benzopyran-4-one derivatives for 6 hrs.

FIG. 22(a) illustrates the effects of control group.

FIG. 22(b) illustrates the effects of compound I-1.

FIG. 22(c) illustrates the effects of compound II-1.

FIG. 23(a)-23(c) shows that rate of cell cycle progression were treated with benzopyran-4-one derivatives for 9 hrs.

FIG. 23(a) illustrates the effects of control group.

FIG. 23(b) illustrates the effects of compound I-1.

FIG. 23(c) illustrates the effects of compound II-1.

FIGS. 24(a)-24(e) show that number of labeled DNA replication.

FIG. 24(a) illustrates the effects of DMSO group.

FIG. 24(b) illustrates the effects of Hydroxyurea (HU) group.

FIG. 24(c) illustrates the effects of ku55933 group.

FIG. 24(d) illustrates the effects of compound I-1.

FIG. 24(e) illustrates the effects of compound II-1.

FIG. 25 illustrates the percentage of EdU incorporation.

### DETAILED DESCRIPTION OF THE PREFERRED EMBODIMENT

Further embodiments herein may be formed by supplementing an embodiment with one or more element from any one or more other embodiment herein, and/or substituting one or more element from one embodiment with one or more element from one or more other embodiment herein.

### EXAMPLES

The following non-limiting examples are provided to illustrate particular embodiments. The embodiments throughout may be supplemented with one or more detail from one or more example below, and/or one or more element from an embodiment may be substituted with one or more detail from one or more example below.

Ataxia telangiectasia-mutated (ATM) and ATM and Rad3-related (ATR) are 2 members of the phosphoinositide 3-kinase (PI3K)-related protein kinases family that play a central role in DNA damage response (DDR) coordination; they also function in the signaling cascades machinery of cell cycle arrest, DNA repair and transcription, and cell death. While ATM is predominant activated in response to DNA strand breaks, ATR is activated in response to damage arising from ultraviolet (UV) ray or replication block; both kinases activate signaling cascades that involving 2 checkpoint kinases effectors, Chk1 and Chk2, whose roles were previously suggested to be redundant. In contrast to ATM, ATR has been reported to be indispensible for cell growth and for life. ATR-knockout mouse embryos died early due to mitotic catastrophe characterized by incomplete DNA replication and chromosomal fragmentation. Moreover, ATR gene mutations are rarely found in humans. The only mutated variants that can survive are heterozygous or hypomorphic variants. Furthermore, cells derived from patients with Seckel syndrome exhibit cellular features associated with ATR signaling cascades defects. Consistent with this phenotype, seckel-like mouse embryonic cells showed accelerated aging due to replicative stress, exhibiting an accumulation of lethal chromosomal breaks. However, with regard to its role in regulating the replication checkpoint, ATR is activated by most cancer chemotherapeutic agents that target DNA in replicating cells. Therefore, inhibition of ATR signaling cascades is a valid and promising strategy that can improve chemotherapeutic or radiotherapeutic efficiency.

Thus so far, several inhibitors of DDR-related kinases, including Chk1 and Chk2, have been successfully used alone or in combination with each other in clinical trials. Recently, several chemicals that inhibit ATR kinase activity in vitro were used to support the hypothesis that ATR kinase can be targeted to improve cancer therapy. Since most of these studies are in their initial stages, it is imperative to focus more efforts toward investigating strategies to inhibit ATR signaling cascades.

In accordance with an aspect of the present invention, benzopyran-4-one derivatives compound, characteristically with inhibiting of DNA Damage Response (DDR), is provided.

Another aspect of this invention, pharmaceutical composition of benzopyran-4-one derivatives, characteristically with inhibiting of DNA Damage Response (DDR), is provided. The benzopyran-4-one derivatives includes a common structure being the following formula I or formula II,

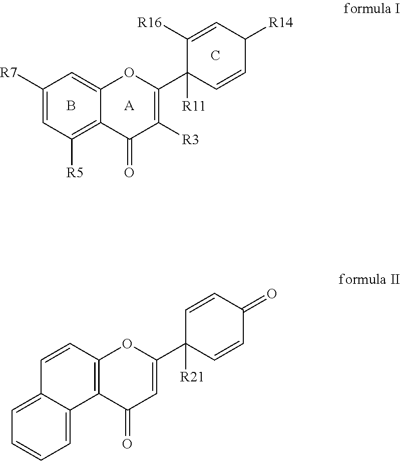

wherein: R₃, R₅, R₇, R₁₁, R₁₄ and R₁₆ are selected independently from a group consisting of a hydrogen, a hydroxyl group, a methoxyl group and a oxygen atom contain a double bond. R₂₁ is selected independently from a group consisting of a hydrogen, a hydroxyl group and a methoxyl group.

In a further aspect of this invention is directed towards a method of treating cancer in a subject in need thereof, including the sequential or simultaneous co-administration of a compound of benzopyran-4-one derivatives or a pharmaceutically acceptable salt thereof, and a DNA-damaging agents. In some embodiments, the DNA-damaging agents are selected from chemotherapeutic drugs such as alkylating agents, antimetabolic agents, antibiotic anti-cancer agents, Topoisomerase inhibitors and anti-mitosis agents.

In some embodiments, the alkylating agent is one selected from Nitrogen mustards (eg. Melphalan, mechlorethamine, Chlorambucil, Ifosfamide, Cyclophosphamide, Estramustine and phenoxybenzamine); or Aziridines (eg. Thiotepa, Carboquone); or Nitrosoureas (eg. Carmustine, Semustine, Iomustine, Nimustine, Streptozocin, Ranimustine and Lomustine); or Procarbazine and triazenes (eg. Dacarbazine, Temozolomide and Procarbazine); or Alkyl sulfonate (eg. Busulfan); or Platinum coordination complex (eg. Cisplatin, Carboplatin, Nedaplatin, Iproplatin and Oxaliplatin); and mixtures thereof.

In some embodiments, the antimetabolic agent is one selected from Thymidylate synthase inhibitor (eg. Aminopterin, Methotrexate, Tegafur, Piritrexin, Trimetrexate, Floxuridine, Raltitrexed, Pemetrexed, Fluorouracil, Doxifluridine and Capecitabine); or Amidophosphoribosyl transferase inhibitors (eg. Mercaptopurine, Thioguanine and Thionosine); or DNA chain elongation inhibitors (eg. Cytarabine, Ancitabine, Gemcitabine, Fludarabine, Cladribine, Clofarabine, Azaserine, Azacitidine, Pentostatin, Hydroxyurea); and mixtures thereof.

In some embodiments, the antibiotic anti-cancer agent is one selected from free radical agents (eg. Bleomycin and Actinomycin D); or Topoisomerase II inhibitors (eg. Daunorubicin, Doxorubicin, Idarubicin, Epirubicin, valrubicin, Pirarubicin, Aclarubicin, Mitoxantrone and Piroxanthrone); or other therapies or anticancer agents (eg. Menogaril, Plicamycin, Acivicin, Anthramycin, Pentostatin, Calicheamicin and Peplomycin); and mixtures thereof.

In some embodiments, the Topoisomerase inhibitor is one selected from Topoisomerase I inhibitors (eg. Camptothecin, Irinotecan, Topotecan); or Topoisomerase II (eg. Podophyllin, Podophyllotoxin, Etoposide, Teniposide); and mixtures thereof.

In some embodiments, the anti-mitosis agent is one selected from Paclitaxel and Docetaxel; or anti-microtubule agents (eg. Colchicine, Vinblastine, Vincristine, Vindesine and Vinorelbine); and mixtures thereof.

Benzopyran-4-one derivatives compounds of this invention include Protoapigenone (I-1), 5′,6′-dihydro-6′-methoxy-protoapigenone (I-2), Protoapigenin, (I-3), protoflavonone (I-4), 5-hydroxyprotoflavone (I-5), 5-hydroxy-7-methoxy-protoflavonone (I-6), compounds I-7, compounds I-8, 3-(1-hydroxy-4-oxocyclohexa-2,5-dienyl)-1-H-benzo[f]chromen-1-one (II-1) and compounds II-2.

In a further aspect of this invention, pharmaceutical composition of benzopyran-4-one derivatives, characteristically with modulating the activation state of ATM kinase is provided. The benzopyran-4-one derivatives includes a common structure being the following formula I or formula II,

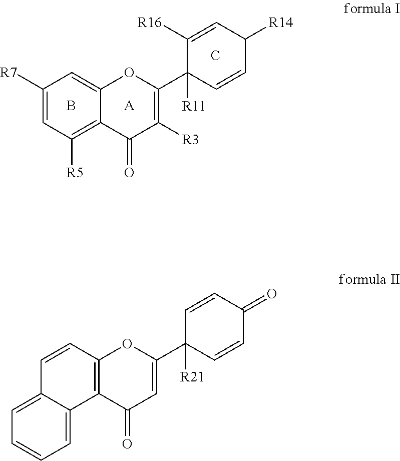

In a further aspect of this invention, both assay kit and assay composition of benzopyran-4-one derivatives, characteristically with detecting the activation state of ATR, DNA DDR kinase signaling cascades is provided.

In a further aspect of this invention is directed towards a method of analyzeing ATR, DNA DDR kinase signaling cascades in a reaction site thereof, including the sequential or simultaneous of benzopyran-4-one derivatives compound or a pharmaceutically acceptable salt thereof, and a chemotherapeutic drugs or additional agents. In some embodiments, the chemotherapeutic drug is selected from chemotherapeutic drugs such as alkylating agents, antimetabolic agents, antibiotic anti-cancer agents, Topoisomerase inhibitors and anti-mitosis agents.

In some embodiments, the individual components of the combination may be administered separately, at different times during the course of therapy, or concurrently, in divided or single combination forms. Also provided is, for example, simultaneous, staggered, or alternating treatment.

In a further aspect of this invention, compounds and pharmaceutical composition of benzopyran-4-one derivatives, characteristically with detecting of DNA damage in cancer cell as determined by the activation state of ATM kinase is also useful for monitoring therapeutic effects during treatment.

In some embodiments, method using benzopyran-4-one derivatives compound or pharmaceutical composition for defecting in the ATR signaling cascade and/or DNA-damage response (DDR). In some embodiments, the defect is altered expression or activity of one or more of the following cell markers as determined by standard cell marker detection assays: ATM, CHK1, CHK2, cellular tumor antigen p53, Adenosine monophosphate activated protein kinase (AMPK), mammalian target of rapamycin complex (mTORC) 1, metal response element (MRE) 11, mitogen-activated protein kinase (MAPK), MAPK-activated protein kinase (MAPKAPK) 2, DNA Repair Protein (RAD50), Nijmegen breakage syndrome (NBS) 1, 53BP1, mediator of DNA damage checkpoint (MDC) 1, H2A histone family member X (H2AX).

In another embodiment, the cell is a cancer cell expressing DNA damaging oncogenes. In some embodiments, the cancer cell has altered expression or activity of one or more of the following cell markers as determined by standard cell marker detection assays: K-Ras, N-Ras, H-Ras, Raf, Myc, Mos, E2F, Cdc25A, CDC4, CDK2, Cyclin E, Cyclin A and Rb.

In a further embodiment, the invention relates to an assay kit or assay composition for determent of ATR and/or DNA DDR signaling cascades at reaction site. In particular the assay kit or assay composition can include, a benzopyran-4-one derivatives compound, a processing/handling plan, a compartment, a additional reagent and instructions for use, or a reagent with a compartment and instructions for use. In one embodiment, for the purpose of altered expression or activity can then generate a detectable at the reaction site of the immunocomplex.

The additional reagent can include ATR, the ATR receptor, the complex of DNA, or an antigenic fragment thereof, a binding composition, or a nucleic acid. A kit for determining the binding of a test compound, e.g., acquired from a biological sample or from a chemical library, can include a control compound, a labeled compound, and a method for separating free labeled compound from bound labeled compound and a combination thereof.

The term excipients or “pharmaceutically acceptable carrier or excipients” and “bio-available carriers or excipients” above-mentioned include any appropriate compounds known to be used for preparing the dosage form, such as the solvent, the dispersing agent, the coating, the anti-bacterial or anti-fungal agent and the preserving agent or the delayed absorbent. Usually, such kind of carrier or excipient does not have the therapeutic activity itself. Each formulation prepared by combining the derivatives disclosed in the present invention and the pharmaceutically acceptable carriers or excipients will not cause the undesired effect, allergy or other inappropriate effects while being administered to human. Accordingly, the derivatives disclosed in the present invention in combination with the pharmaceutically acceptable carrier or excipients are adaptable in the clinical usage and in the human. A therapeutic effect can be achieved by using the dosage form in the present invention by the local or sublingual administration via the venous, oral, and inhalation routes or via the nasal, rectal and vaginal routes. About 0.1 mg to 1000 mg per day of the active ingredient is administered for the patients of various diseases.

The carrier is varied with each formulation, and the sterile injection composition can be dissolved or suspended in the non-toxic intravenous injection diluents or solvent such as 1,3-butanediol. Among these carriers, the acceptable carrier may be mannitol or water. Besides, the fixing oil or the synthetic glycerol ester or di-glycerol ester is the commonly used solvent. The fatty acid such as the oleic acid, the olive oil or the castor oil and the glycerol ester derivatives thereof, especially the oxy-acetylated type, may serve as the oil for preparing the injection and as the naturally pharmaceutical acceptable oil. Such oil solution or suspension may include the long chain alcohol diluents or the dispersing agent, the carboxylmethyl cellulose or the analogous dispersing agent. Other carriers are common surfactant such as Tween and Spans or other analogous emulsion, or the pharmaceutically acceptable solid, liquid or other bio-available enhancing agent used for developing the formulation that used in the pharmaceutical industry.

The composition for oral administration adopts any oral acceptable formulation, which includes capsule, tablet, pill, emulsion, aqueous suspension, dispersing agent and solvent. The carrier generally used in the oral formulation, taking a tablet as an example, the carrier may be lactose, corn starch and lubricant, and magnesium stearate is the basic additive. The excipients used in a capsule include lactose and dried corn starch. For preparing an aqueous suspension or an emulsion formulation, the active ingredient is suspended or dissolved in oil interface in combination with the emulsion or the suspending agent, and appropriate amount of sweetening agent, flavors or pigment is added as needed.

The nasal aerosol or inhalation composition may be prepared according to the well-known preparation techniques. For example, the bioavailability can be increased by dissolving the composition in the phosphate buffer saline and adding the benzyl alcohol or other appropriate preservative, or the absorption enhancing agent. The compound of the present invention may be formulated as suppositories for rectal or virginal administration.

The compound of the present invention can also be administered intravenously, as well as subcutaneously, parentally, muscular, or by the intra-articular, intracranial, intra-articular fluid and intra-spinal injections, the aortic injection, the sterna injection, the intra-lesion injection or other appropriate administrations.

Protoapigenone (I-1) induces chromosomal aberrations but does not produce marked DDR.

Previously, Protoapigenone (I-1) and compound II-1 were demonstrated to cause DNA strand breaks and apoptosis in lung and prostate cancers (Chen H M, et al., Free Radic Biol Med 2011), suggesting that inducing DNA damage may be the potential mechanism underlying the anticancer effect of benzopyran-4-one derivatives.

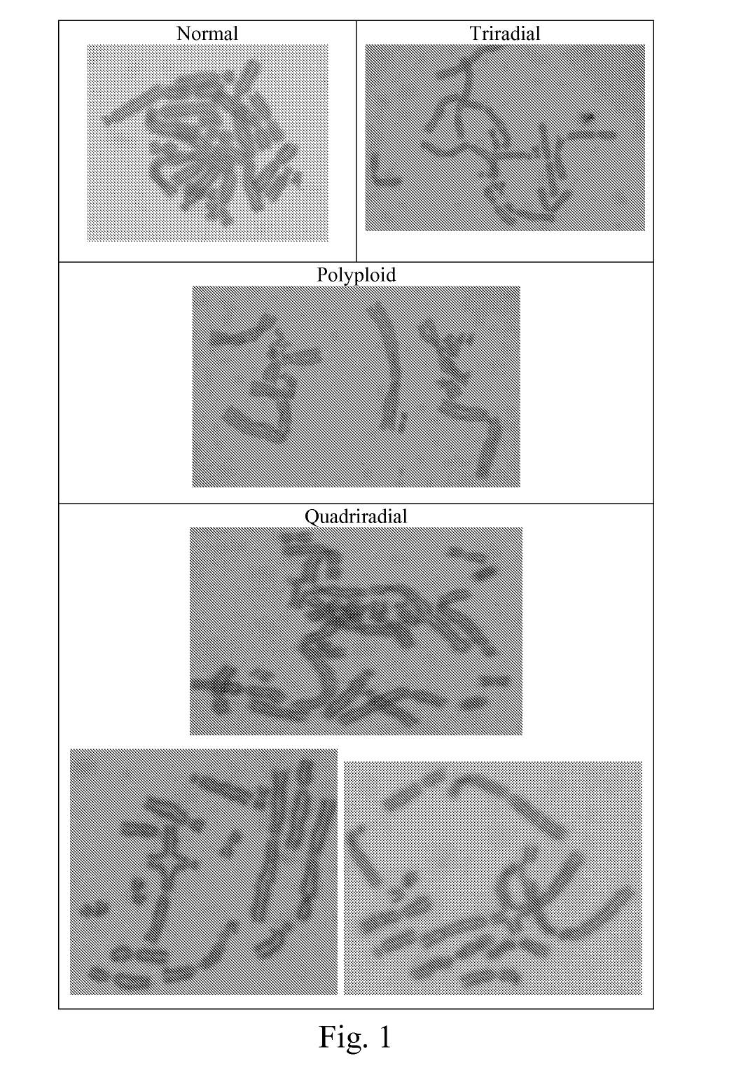

To test this hypothesis, it is investigated the cytogenetic effect of Protoapigenone (I-1) on CHO cells (FIG. 1). According to the Table 1, low Protoapigenone (I-1) concentrations produced dose-dependent increases in chromosomal structural changes, such as breakages, radials, and chromosomal polyploidy, similar to the effects seen with mitomycin C treatment; however, the complete mitotic chromosome could not be obtained upon high-dose Protoapigenone (I-1) treatment.

TABLE 1
Protoapigenone (I-1) induces chromosomal aberration in CHO cells

| | DMSO | Protoapigenone | | mitomycin |
|---|---|---|---|---|
| Treatment | control | (I-1) | | C |
| Concentration (μM) | 0.00 | 2.17 | 4.35 | 2.00 |
| chromatid break (No.) | 0 | 2 | 1 | 2 |
| Chromatid deletion | 0 | 0 | 1 | 3 |
| Triradial | 0 | 4 | 13 | 42 |
| quadriradial | 0 | 3 | 9 | 29 |
| ring | 0 | 1 | 2 | 0 |
| complex rerrangement | 0 | 0 | 0 | 2 |
| dicentric | 0 | 0 | 0 | 1 |
| polyploid | 1 | 3 | 1 | 0 |
| pulverized cell | 0 | 0 | 1 | 5 |
| Average aberrant | 0.5 | 6.5 * | 14.0 * | 42.0 * |
| metaphases (%)^{a} | | | | |

Note:
1. Two hundred cells per treatment were analysized for chromosomal aberration.
2. Type of structural aberrations, such as chromatid break, chromatid deletion, triradial, quadriradial, ring, complex rerrangement, dicentric, polyploid and pulverized cell numbers (No.) were indicated.
3. Others chromosome gap, chromosome break, chromosome deletion and chromatid gap were not be observed in this experiment.
4. ^{a}, * indicated statistic significantly for tested vs. control group by t-test.

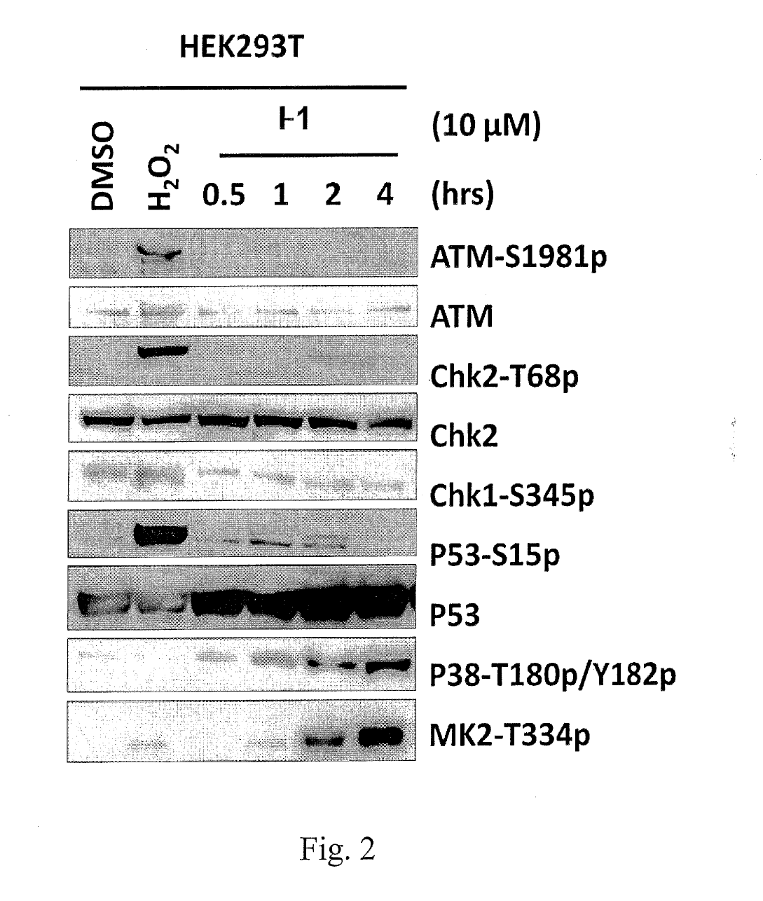

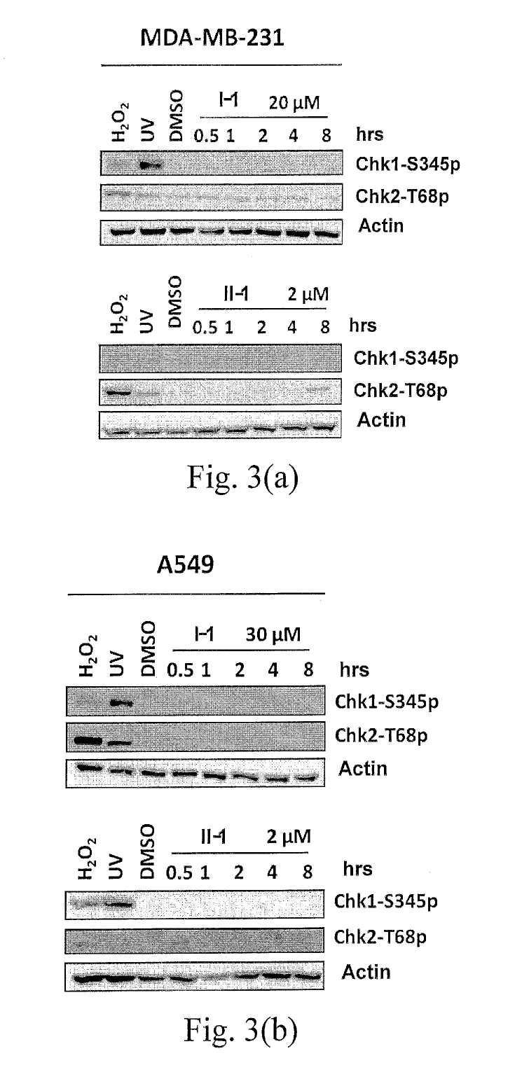

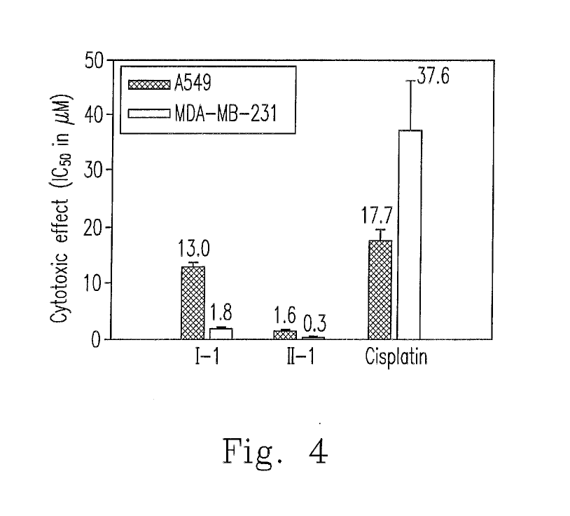

Since mitomycin C can induce DDR in many cancers, it is investigated what kind of DDR signaling was activated by Protoapigenone (I-1). Surprisingly, high doses of Protoapigenone (I-1) in HEK293T cells did not induce noticeable changes in the putative DDR signaling, which it is measured by analyzing the phosphorylation of the ATM-dependent Chk2 Thr⁶⁸ residue and the ATR-dependent Chk1 Ser³⁴⁵ residue (FIG. 2). It did observe that Protoapigenone (I-1) treatment caused slight accumulation of the p53 protein, which could have been the result of several posttranslational modifications. However, phosphorylation of the p53 Ser¹⁵ residue did not contribute to this Protoapigenone (I-1)-induced p53 protein accumulation, suggesting that Protoapigenone (I-1) does not directly damage DNA because DNA damage normally stimulates ATM/ATR-dependent p53 Ser15 phosphorylation. Our result is similar to previous reports that p38 MAPK is activated by Protoapigenone (I-1) (Chen W Y, et al. Invest New Drugs 2011), as its downstream target MAPKAPK2 was found to be phosphorylated starting as early as 2 h after Protoapigenone (I-1) exposure (FIG. 2). It is repeated the benzopyran-4-one derivatives experiment on lung and breast carcinoma cell lines A549 and MDA-MB-231 cells, respectively, and obtained similar results. Consistently, no marked changes in Chk1 and Chk2 phosphorylation signaling were detected even at high doses of either drug for as long as 8 h after drug treatment (FIGS. 3(a) and 3(b)). The cytotoxic effect by benzopyran-4-one derivatives on cancer cells was determined by MTT assay at 48 h of incubation (FIG. 4); our data indicated that the IC₅₀ value range for cytotoxicity was similar to those in previous reports, confirming that benzopyran-4-one derivatives are stable compounds that do not directly cause DNA damage.

Protoapigenone (I-1) and compound II-1 inhibit Chk1 phosphorylation after DNA damage.

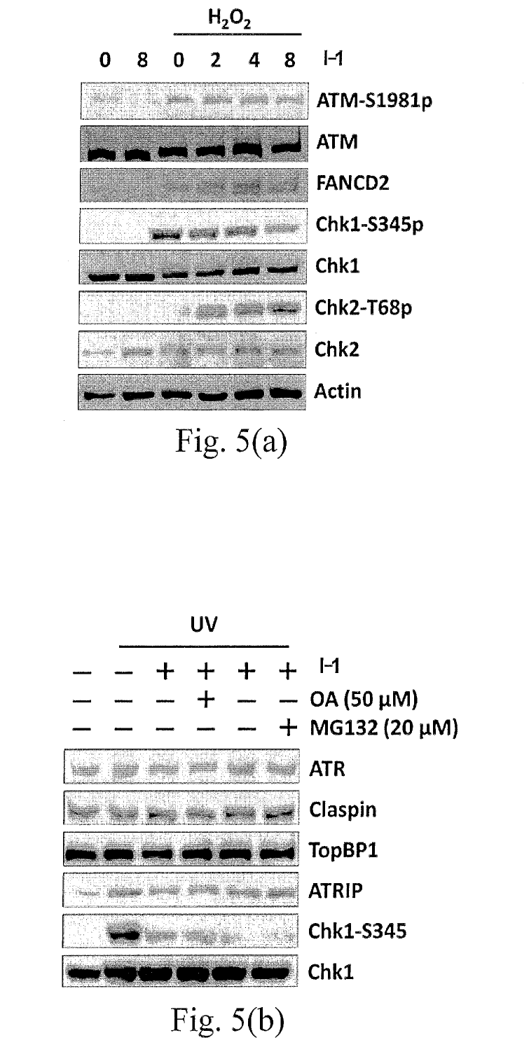

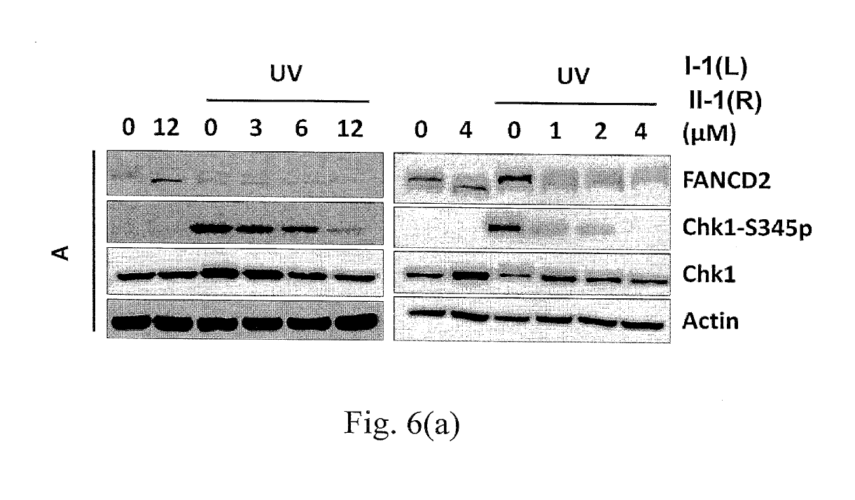

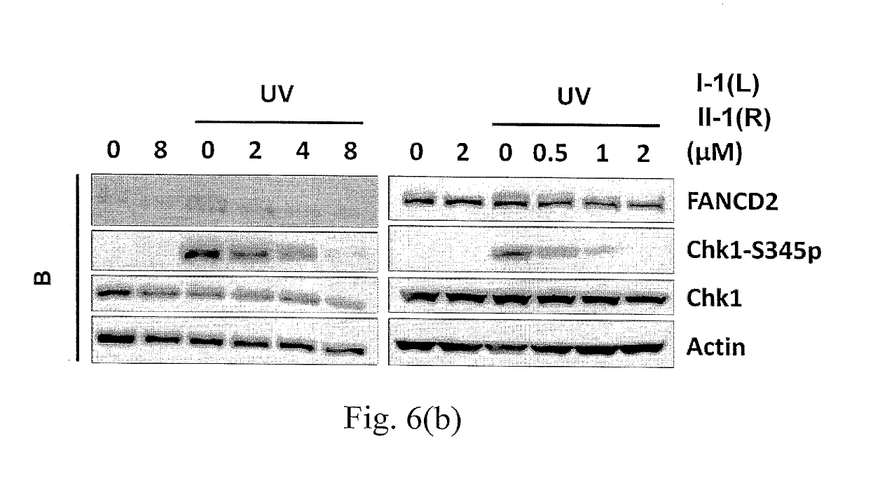

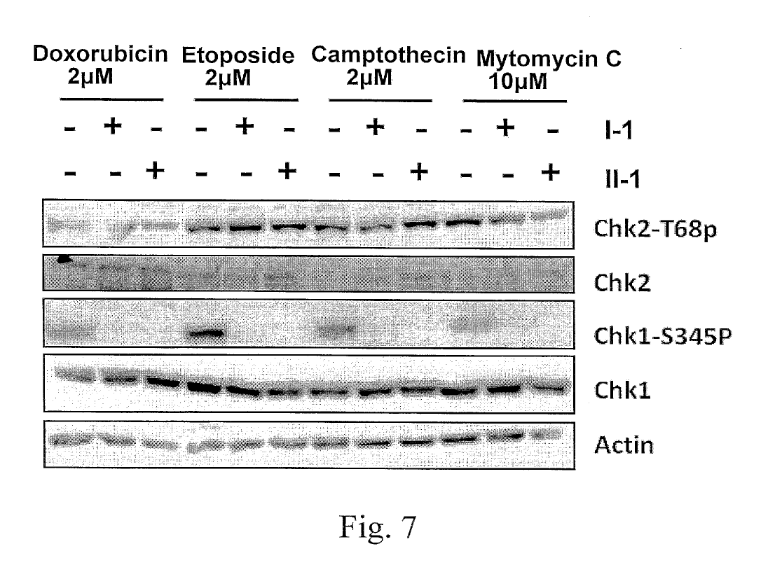

Understanding the mechanism by which the benzopyran-4-one derivatives compounds cause chromosomal breakages (FIG. 1) and other abnormalities might aid in identifying their targets. It is hypothesized that genes with functions associated with DNA damage checkpoints and/or DNA repair might be targeted by benzopyran-4-one derivatives. To test this hypothesis, it is assessed the effects of benzopyran-4-one derivatives on DDR induced by H₂O₂. Protoapigenone (I-1) was found to inhibit Chk1, but promote Chk2 phosphorylation in A594 cells treated with 0.1 mM H₂O₂ for 2 h; however, ATM autophosphorylation was not affected (FIG. 5(a)). Pretreatment of cells with okadaic acid (OA) (a phosphatase inhibitor) or MG132 (a proteasome inhibitor) could not reverse the Protoapigenone (I-1)-induced inhibition of Chk1 phosphorylation, indicating that the inhibition does not occur due to phosphatase activation or proteasome degradation by other regulatory factors (FIG. 5(b)). Further, it is investigated other sources of DNA stimuli specific for ATR activation; our results demonstrate that UV-induced Chk1 phosphorylation was dose-dependently inhibited by benzopyran-4-one derivatives within different cells (FIGS. 6(a) and 6(b)). In response to DNA double-strand breaks (DSBs), FANCD2 is known to be monoubiquitinated on K561 (FANCD2-Ub) in an ATR-dependent manner to stimulate repair (Andreassen P R, et al. Genes Dev 2004). It is showed that FANCD2-Ub was also inhibited by benzopyran-4-one derivatives (FIGS. 5(a), 6(a) and 6(b)); further, ATR inhibition by benzopyran-4-one derivatives was also observed in cells treated with currently prescribed chemotherapeutic agents (FIG. 7). Collectively, these findings indicate that benzopyran-4-one derivatives can modify ATR signaling after various types of DNA damage. Interestingly, compound II-1 was more potent than Protoapigenone (I-1) in inhibiting Chk1 phosphorylation and cytotoxicity (FIGS. 4, 6(a) and 6(b)).

It is speculated that the replacement of 2 hydroxyl groups on Protoapigenone (I-1) with an additional benzene ring contributes positively to this ATR inhibition; however, the definite pharmacophores need to be further investigated when the ATR protein structure is resolved.

Target specificity of Protoapigenone (I-1) and compound II-1 for ATR-mediated signaling inhibition.

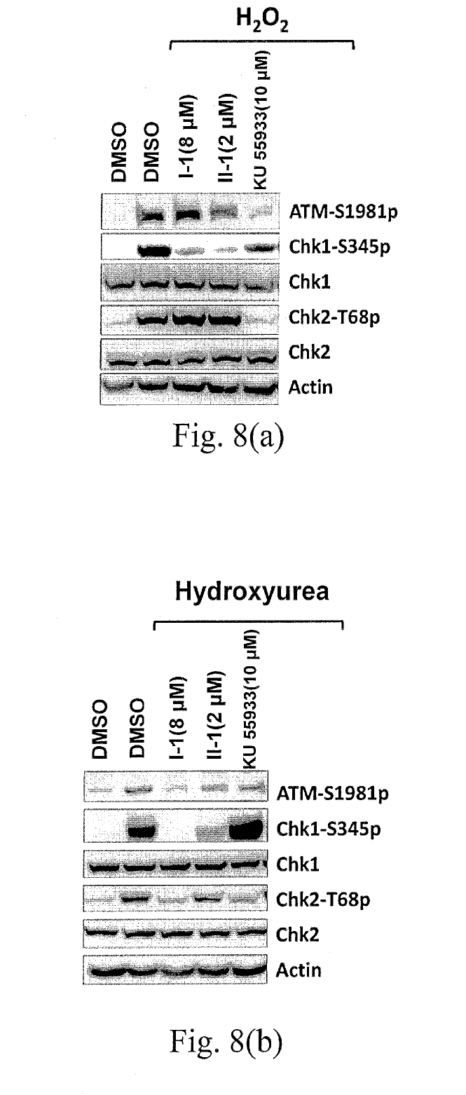

To elucidate the specificity of the benzopyran-4-one derivatives inhibition on ATR-mediated signaling, it is compared the change between cells treated with benzopyran-4-one derivatives or the ATM-specific inhibitor KU55933 before the induction of DDR. After H₂O₂ damage, ATM is thought to be the principal responder, and KU55933 treatment strongly inhibited ATM-mediated Chk2 phosphorylation specifically, but its effect on ATR-mediated Chk1 phosphorylation was small (FIG. 8(a)). In contrast, after hydroxyurea (HU; a replication blocker) damage, ATR is thought to be the principal responder, and benzopyran-4-one derivatives treatment significantly inhibited Chk1 phosphorylation, but only slightly inhibited Chk2 phosphorylation (FIG. 8(b)).

Using these pharmacological methods, it is demonstrated that the specificity of DDR inhibition between benzopyran-4-one derivatives and KU55933 was completely different. To strengthen the argument that benzopyran-4-one derivatives specifically inhibits ATR signaling, small inhibitory RNAs against ATM, ATR, and the catalytic subunit of DNA protein kinase (DNA-PKcs) were introduced into HEK293T cells before exposure to UV or H₂O₂.

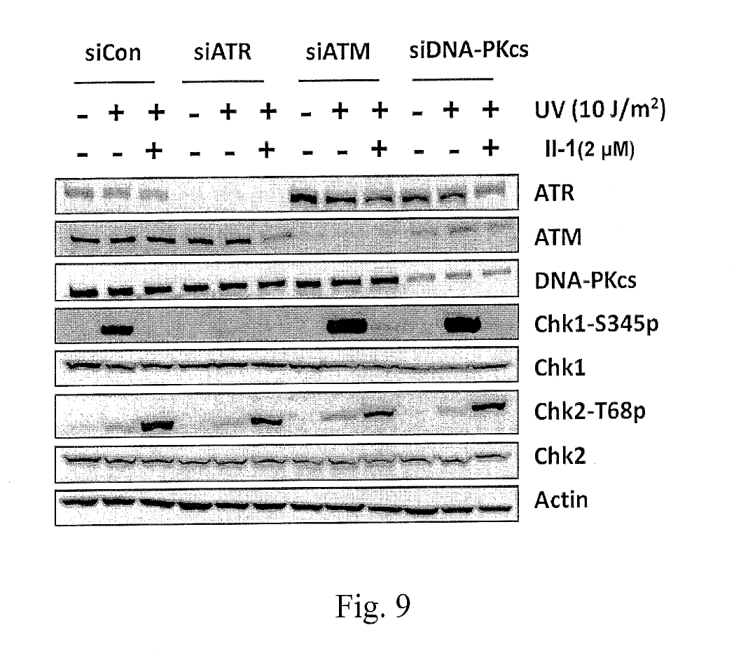

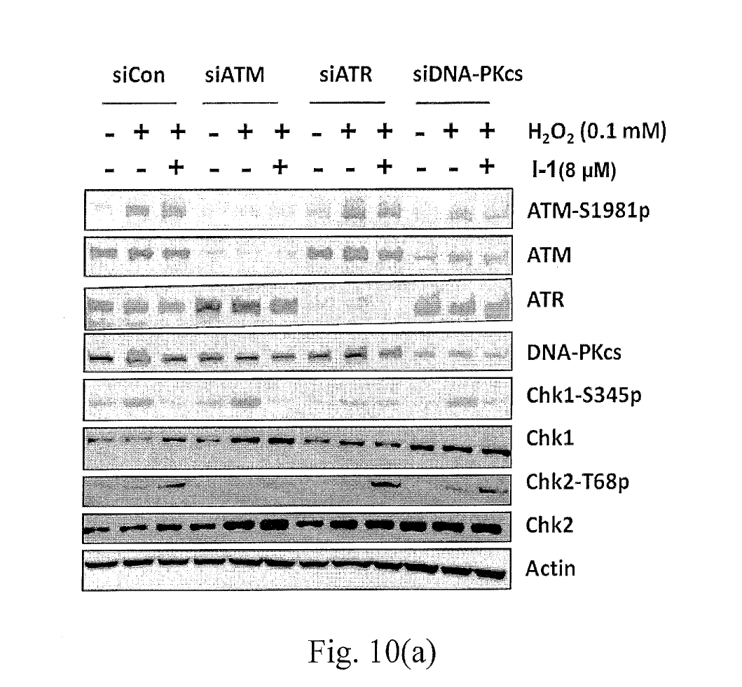

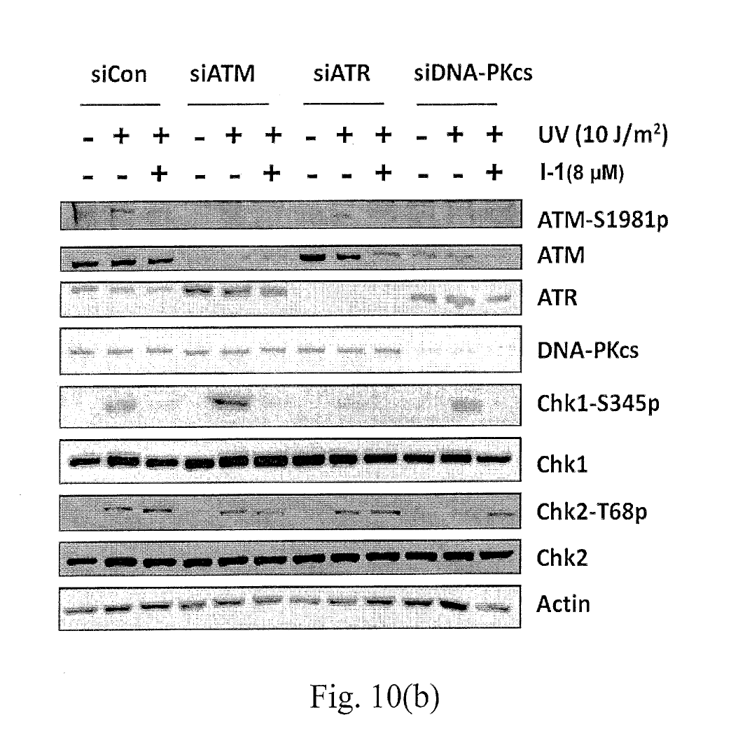

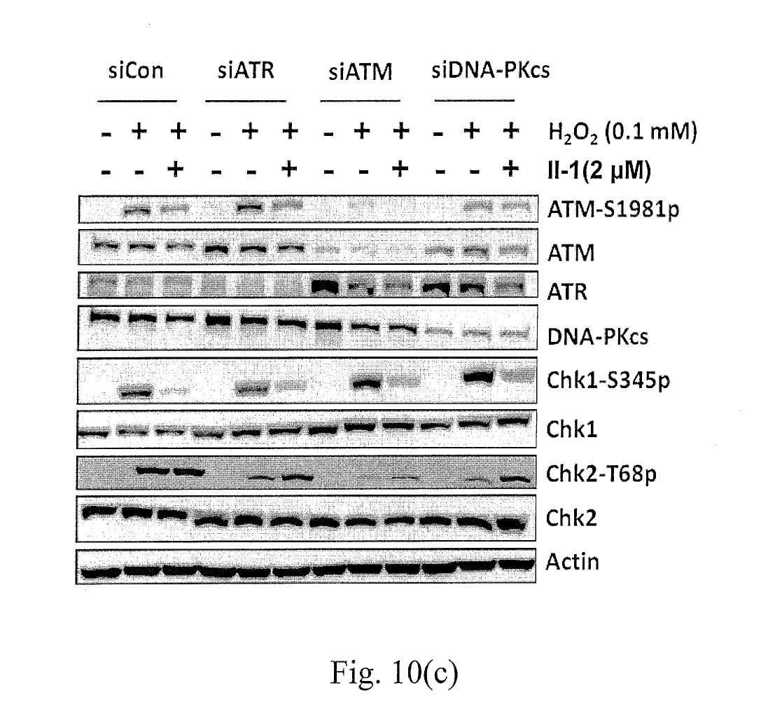

Our results demonstrated that benzopyran-4-one derivatives completely inhibited UV-induced or H₂O₂-induced Chk1 phosphorylation in a manner similar to siRNA knockdown of ATR, but not ATM or DNA-PKcs (FIG. 9, FIGS. 10(a), 10(b) and 10(c)). The siRNAs against ATM and DNA-PKcs decreased the UV-induced or H₂O₂-induced Chk2 phosphorylation, which were not altered by the addition of Protoapigenone (I-1), but were increased by compound II-1 treatment. Interestingly, neither siRNA targeted to ATM or ATR nor DNA-PKcs affected the compound II-1-mediated increase in Chk2 phosphorylation. Since a high dose of compound II-1 itself slightly induces Chk2 activation (FIGS. 3(a) and 3(b)), the increased Chk2 phosphorylation was likely a synergistic effect due to DNA damage.

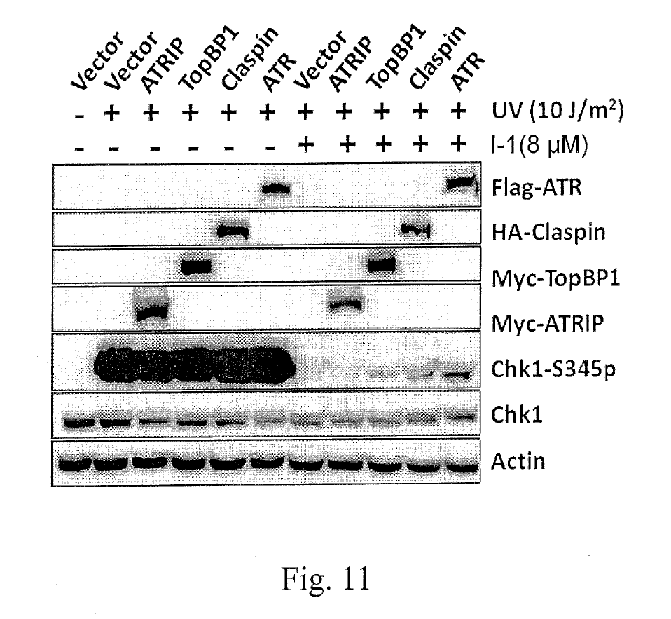

To further identify the specific mediator that contributes to the effect of Protoapigenone (I-1) on the initiation of ATR kinase activation, it is tested whether TopBp1, ATRIP, and claspin were involved, as they have been identified as mediators of ATR kinase activation (Lopez-Contreras A J, et al. DNA Repair (Amst) 2010). Our results demonstrated that overexpression of ATRIP or TopBP1 did not reverse the inhibitory effect of Protoapigenone (I-1) on Chk1 phosphorylation, whereas overexpression of claspin or ATR did (FIG. 11), suggesting that Protoapigenone (I-1) might affect the function of ATR or claspin contributes to ATR signaling inhibition.

Protoapigenone (I-1) and compound II-1 impair the functions of DNA damage checkpoints and DNA repair.

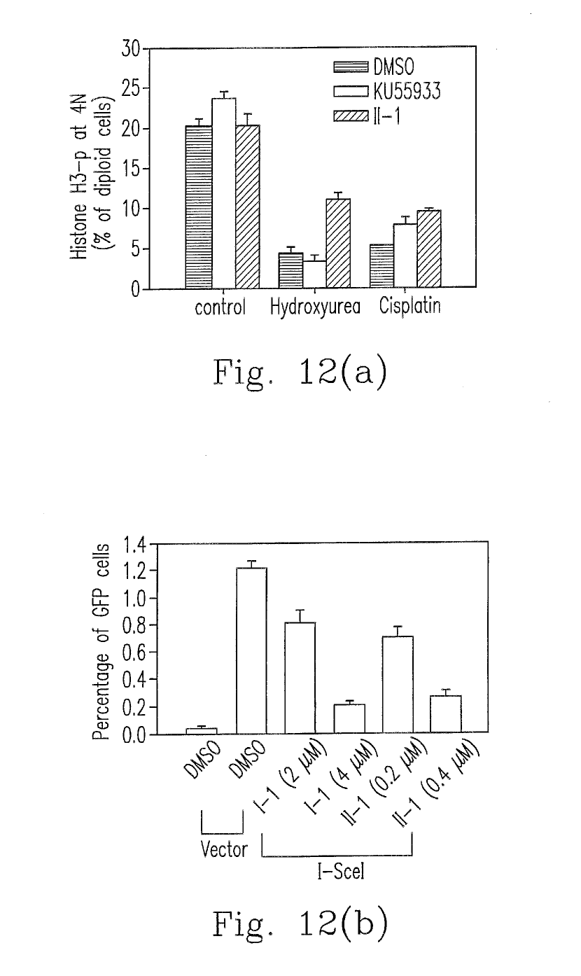

Previously, it has been demonstrated that S/M and G2/M checkpoints are activated by ATR in response to different types of DNA damage (Nghiem P, et al. Proc Natl Acad Sci USA 2001). Of these, the G2/M checkpoint involves ATM and ATR in collaboration, whereas the S/M checkpoint is mediated solely by ATR. To maintain genetic integrity, ATR can prevent premature mitotic entry in the event of incomplete DNA replication or unrepaired DNA damage. In order to evaluate the effect of benzopyran-4-one derivatives on these ATR-associated DNA damage checkpoints, it is observed the effect of benzopyran-4-one derivatives on mitotic entry following hydroxyurea or cisplatin treatment. In MDA-MB-231 cells, hydroxyurea and cisplatin significantly decreased the number of mitotic cells, indicating that the S/M and G2/M checkpoints are intact in MDA-MB-231 cells (FIG. 12(a)).

TABLE 2
The percentage of mitotic cells

| | Control | Compound | | |
|---|---|---|---|---|
| | group | I-1 | II-1 | KU55933 |
| Control | 20.28 ± 0.86 | 23.69 ± 0.94 | 12.05 ± 0.68 | 20.29 ± 1.48 |
| group | | | | |
| hydroxy- | 4.46 ± 0.67 | 3.40 ± 0.41 | 10.49 ± 1.17 | 11.19 ± 0.76 |
| urea | | | | |
| cisplatin | 5.35 ± 0.07 | 7.99 ± 0.91 | 8.14 ± 0.99 | 9.70 ± 0.25 |

Benzopyran-4-one derivatives or KU55933 treatment increased the percentage of mitotic cells in cisplatin-treated cells, as the Table 2 suggesting that all of these compounds inhibited the damage-induced G2/M checkpoint. However, benzopyran-4-one derivatives, but not KU55933, significantly increased the HU-induced mitotic entry that is specific for the S/M checkpoint, indicating that benzopyran-4-one derivatives specifically impaired this distinctive checkpoint mediated solely by ATR (FIG. 12(a)).

ATR function is also linked to DNA repair via its coupled targets (Sorensen C S, et al. Nat Cell Biol 2005). To examine the effect of benzopyran-4-one derivatives treatment on DNA repair, it is performed a homologous recombination repair (HRR) assay in HeLa cells.

TABLE 3
The percentage of GFP cell (DNA homologous
recombination repair assay)

| treatment | | | GFP cell (%) |
|---|---|---|---|
| Un-treatment group | | 0.0 μM | 0.040 ± 0.0097 |
| chromosomal breaks | Un-treatment | 0.0 μM | 1.217 ± 0.0203 |
| generated by I-SceI | Compound I-1 | 2.0 μM | 0.807 ± 0.0403 |
| endonuclease | | 4.0 μM | 0.213 ± 0.0105 |
| expression group | Compound II-1 | 0.2 μM | 0.703 ± 0.0304 |
| | | 0.4 μM | 0.017 ± 0.0169 |

Our result, as Table 3 demonstrated that chromosomal breaks normally repaired by HRR were dose-dependently inhibited by Protoapigenone (I-1) at low concentrations. Compound II-1 produced similar effects at doses that were 10-fold lower than that of Protoapigenone (I-1) (FIG. 12(b)). From these results, it is assumed that the cells carrying unrepaired DNA would enter into mitosis following DNA damage. To verify this assumption, it is analyzed the DNA-damage marker gamma-H2AX on mitotic cells using immunofluorescence staining. As expected, the numbers of large gamma-H2AX foci were increased upon addition of benzopyran-4-one derivatives in both unperturbed and perturbed mitotic cells (FIG. 13), suggesting that benzopyran-4-one derivatives increase DNA damage in mitotic cells. The chromosomes became flat and aggregated after benzopyran-4-one derivatives treatment, differing from the three-dimensional and hair-like appearance of normal chromosomes at metaphase.

Protoapigenone (I-1) and compound II-1 enhance chemosensitivity.

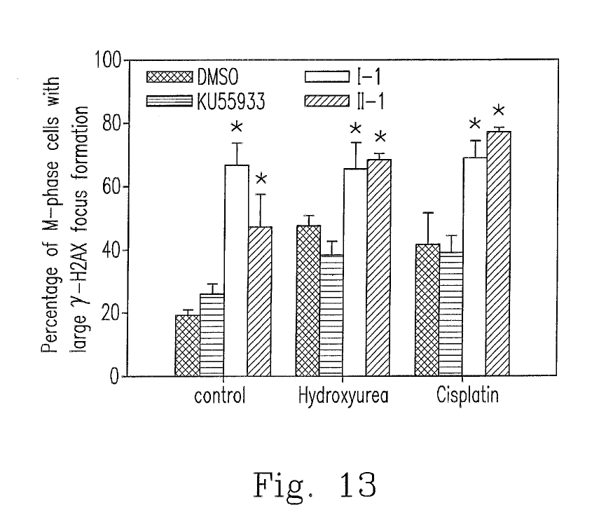

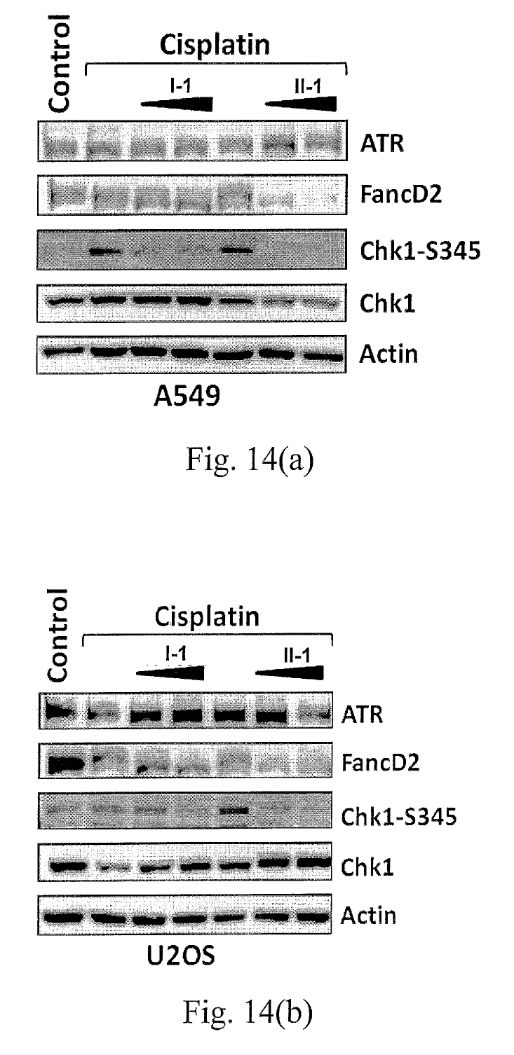

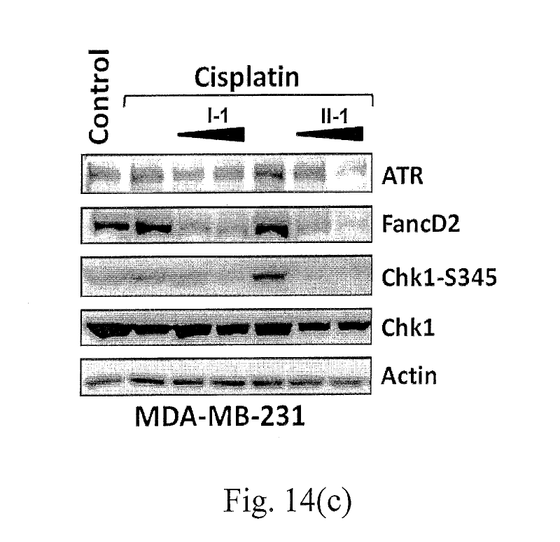

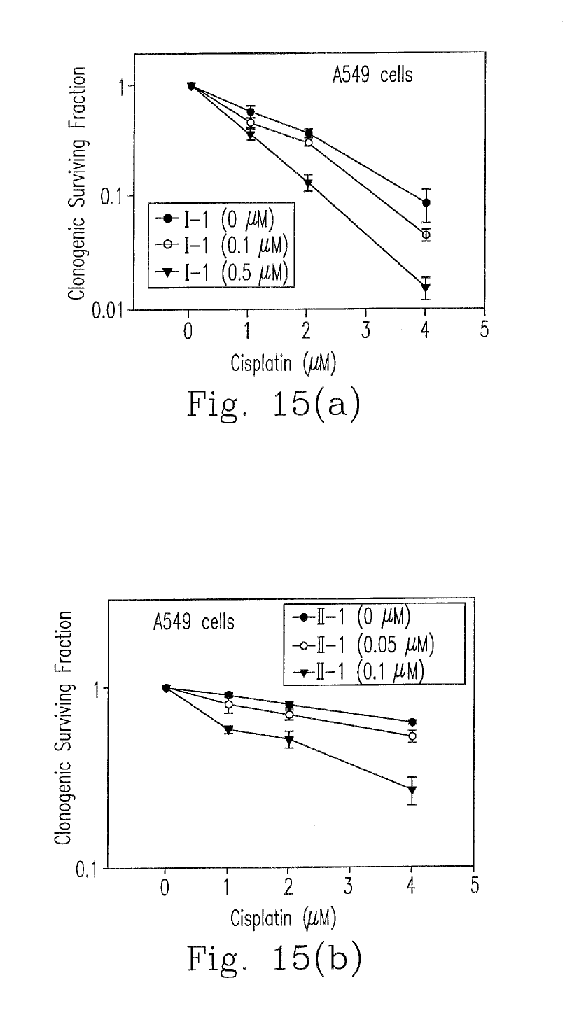

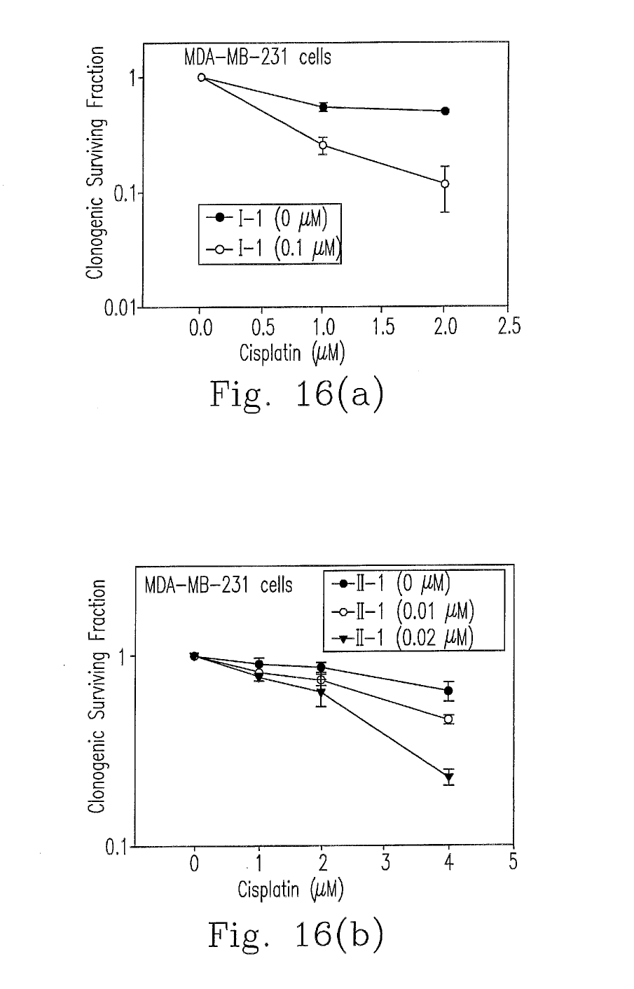

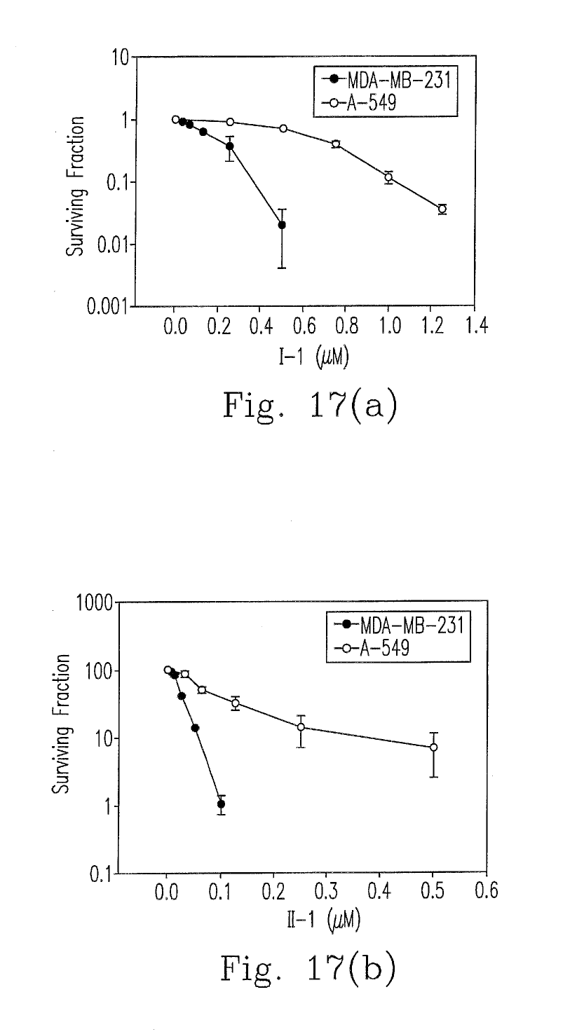

Inhibition of the checkpoint and repair mechanisms leads to chemosensitization in cancers. It is questioned whether benzopyran-4-one derivatives could function as sensitizers for the chemotherapeutic drugs cisplatin that has been shown to induce ATR activation as well as FANCD2 monoubiquitination, which is the vital step for DNA crosslink repair (Chirnomas D, et al. Mol Cancer Ther 2006). It is found that benzopyran-4-one derivatives treatment decreased the cisplatin-induced Chk1 phosphorylation and FANCD2 monoubiquitination in A549, MDA-MB-231, and U2OS cells (FIGS. 14(a), 14(b) and 14(c)). Using the same doses, compound II-1 not only inhibits monoubiquitination of FANCD2 but also affects FANCD2 protein stability; this data emphasizes that compound II-1 has more potent inhibitory effects as compared to Protoapigenone (I-1). It is further treated individual cells with cisplatin in combination with several varying doses of benzopyran-4-one derivatives, and counted survival colonies to determine their ability to survive after cisplatin-induced damage. Our results demonstrated that benzopyran-4-one derivatives effectively decreased the clonogenic survival in cisplatin-treated MDA-MB-231 and A549 cells in the nanomolar dose range (FIG. 15(a), 15(b), FIGS. 16(a) and 16(b)). To investigate the chemosensitization effect of low-dose benzopyran-4-one derivatives in vivo, it is established a tumor xenograft in nude mice using human MDA-MB-231 tumor cells, which are considered to be more resistant to cisplatin and are also sensitive to treatment with benzopyran-4-one derivatives, at least as compared to A549 cells (FIG. 4, FIGS. 17(a) and 17(b)). When the mice were treated with 0.2 mg/kg compound II-1 in combination with 2 mg/kg cisplatin, the tumor inhibitory effect was greater than that of cisplatin treatment alone (FIG. 18). However, Protoapigenone (I-1) unexpectedly did not affect the cisplatin sensitivity of MDA-MB-231 tumors when a higher dose of 2 mg/kg was used in our experiments (data not shown). The pharmacokinetic data of Protoapigenone (I-1) and compound II-1 needs to be compared in future studies to determine the differences in the chemical effects of these 2 compounds in vitro and in vivo.

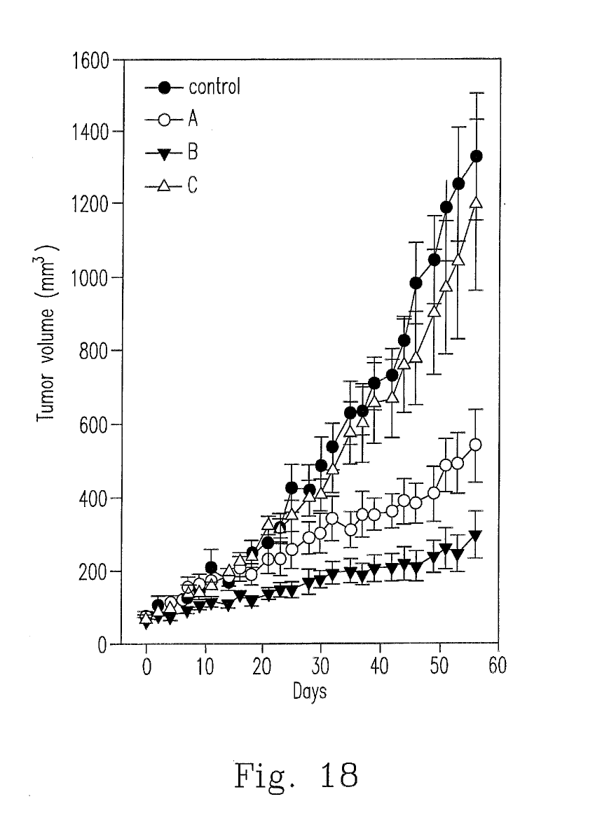

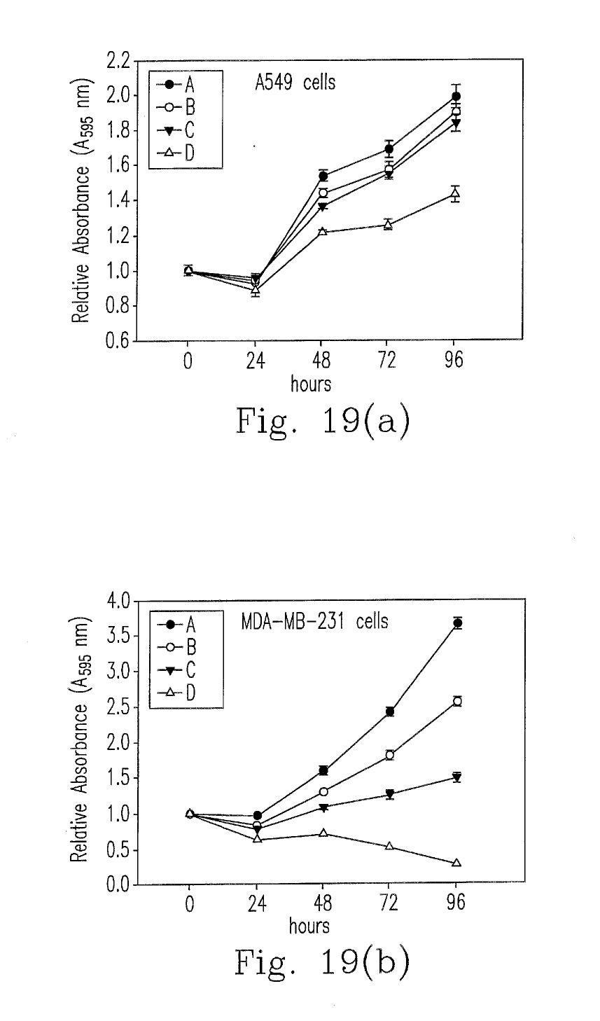

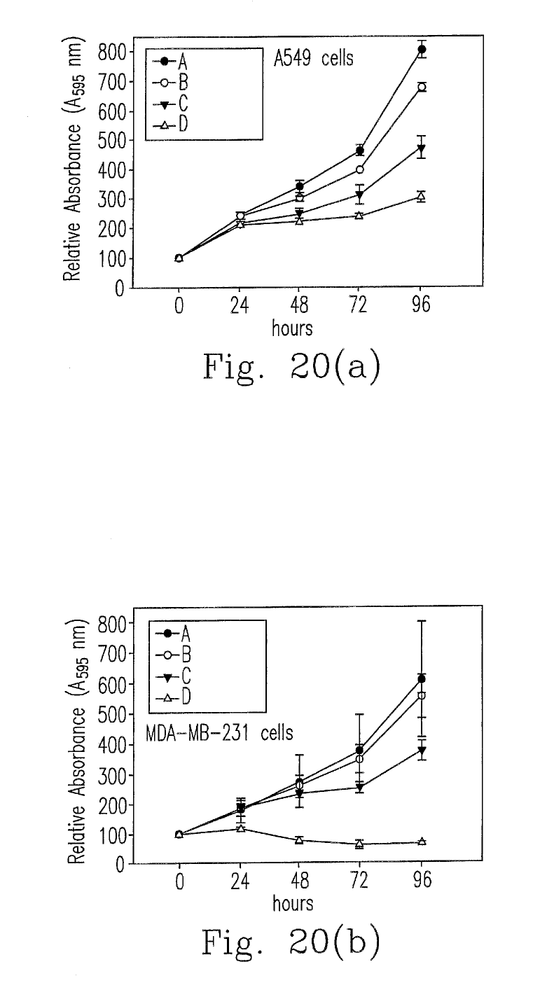

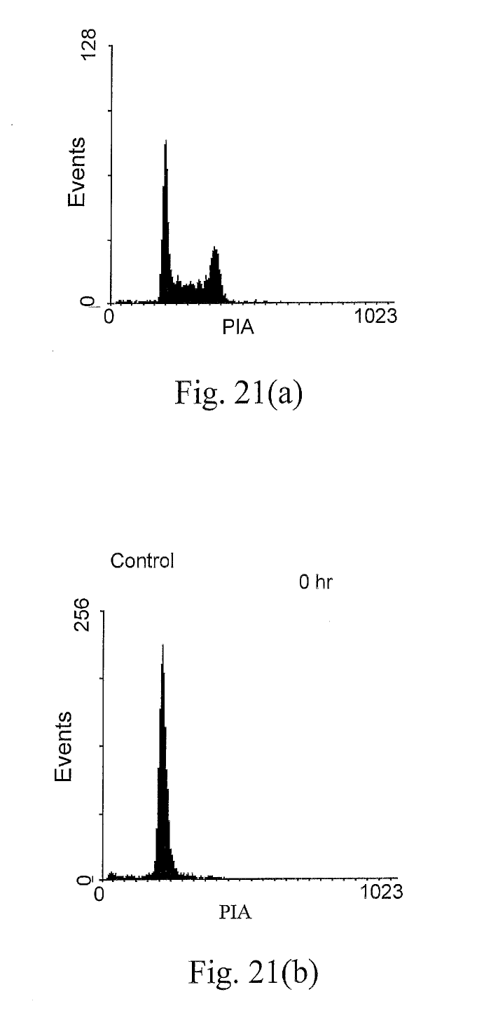

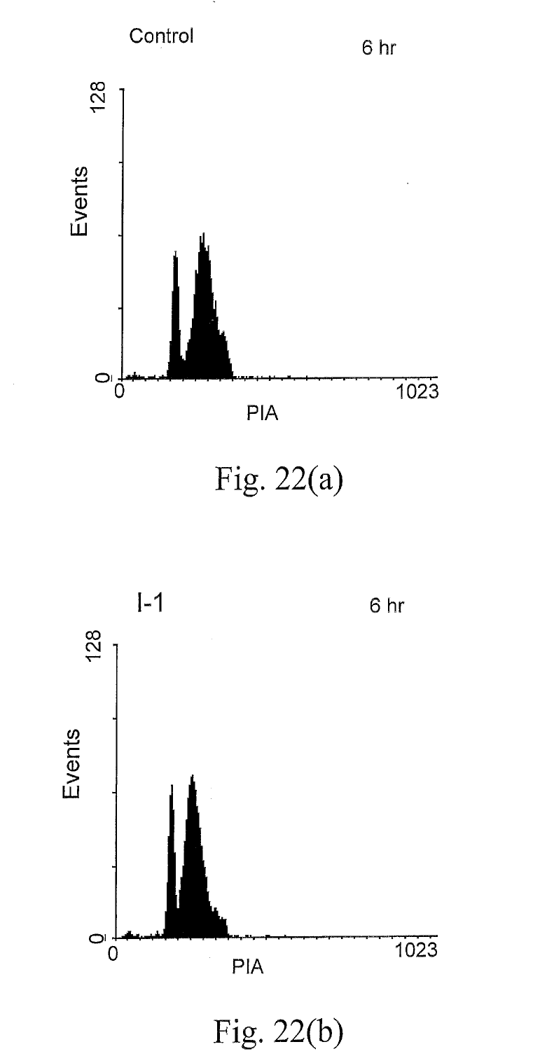

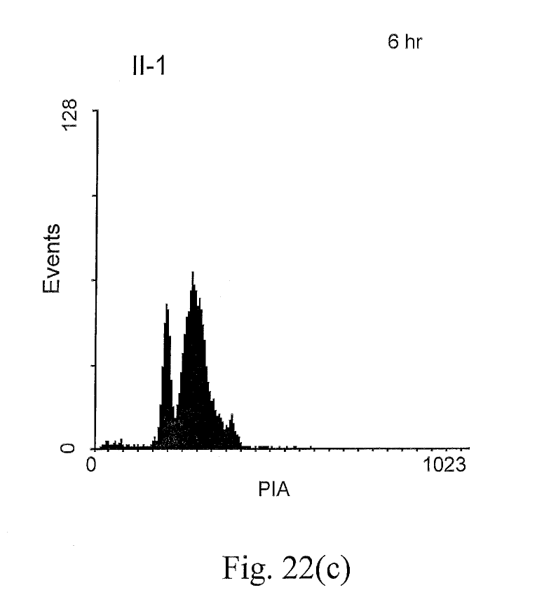

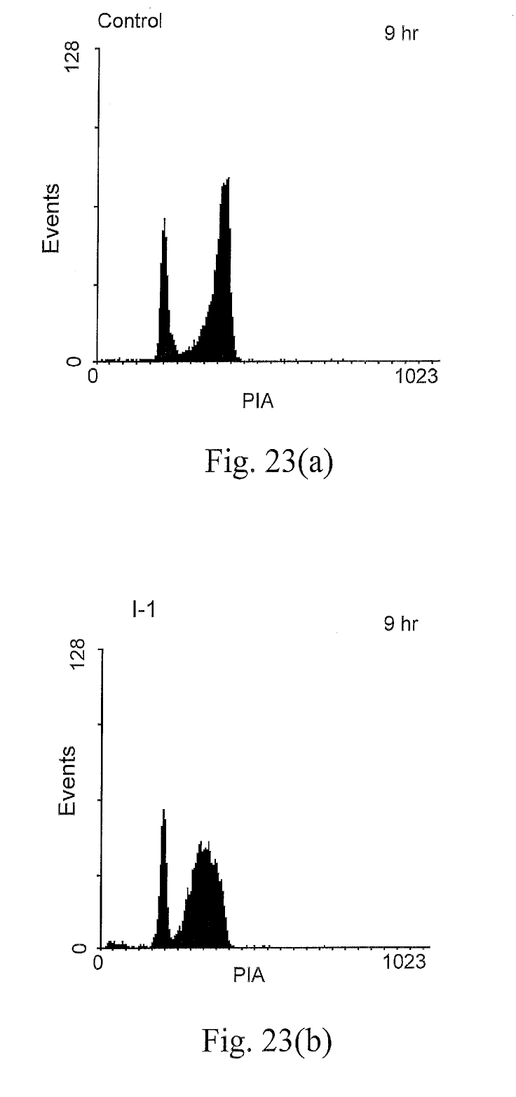

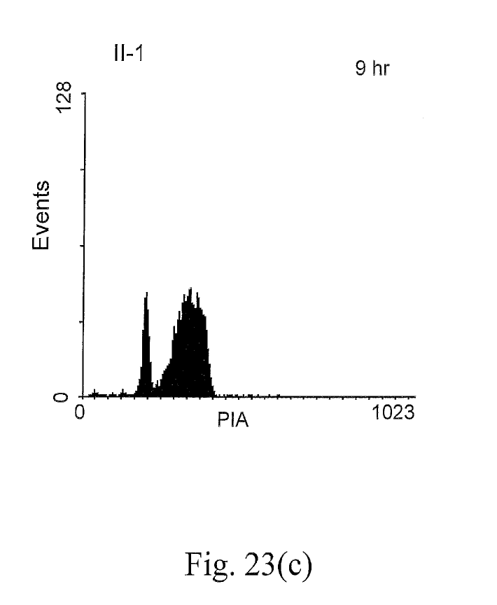

ATR are involved in DNA replication. Low doses of Protoapigenone (I-1) and compound II-1 significantly slowed cancer growth in a dose-dependent manner (FIG. 19(a), 19(b) FIGS. 20(a) and 20(b)), and caused S phase delay and inhibition of DNA synthesis (FIG. 22(a), 22(b), 22(c), FIG. 23(a), 23(b) and 23(c)); these events are similar to previously reported characteristics of ATR defects. In the results of double-thymidine cell cycle synchronization assay, according to the Table 4 which sorted out from FIG. 21-23, the unsynchronized cells (FIG. 21(a)) become synchronization by using this method, and 97% of cells were stopped at G1/S boundary after two cycles of thymidine blocks (FIG. 21(b)). Those synchronized cells released from thymidine blockade and allowed progress into S phase in presence or absence of protoapigenone (I-1) and compound II-1. Protoapigenone (I-1) (FIGS. 22(b) and 23(c)) and compound II-1 (FIGS. 22(c) and 23(c)) showed significantly reduce the percentage of G2/M cells at 9 hours of treatment in compared with control group (FIGS. 22(a) and 23(a)). So far, indicating that benzopyran-4-one derivatives with the ability to delay the S phase progression.

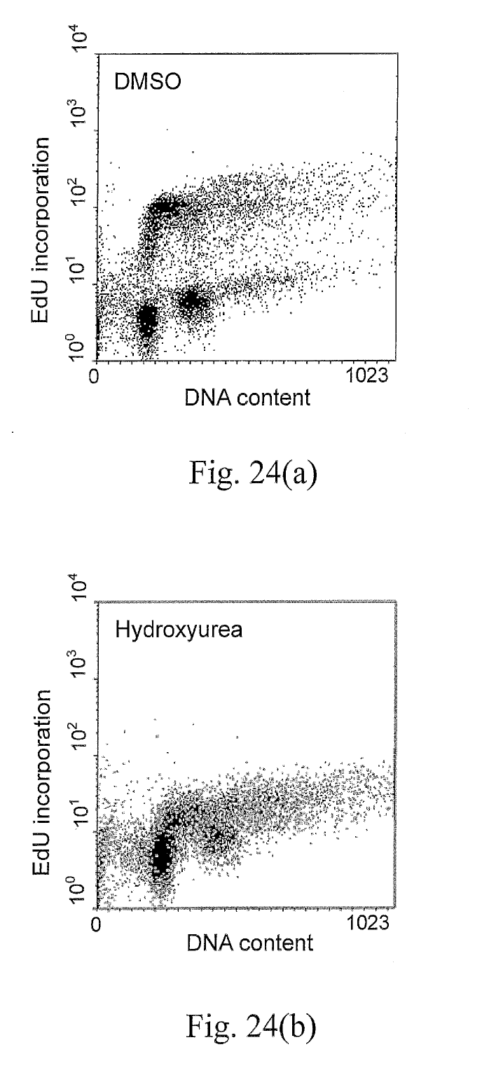

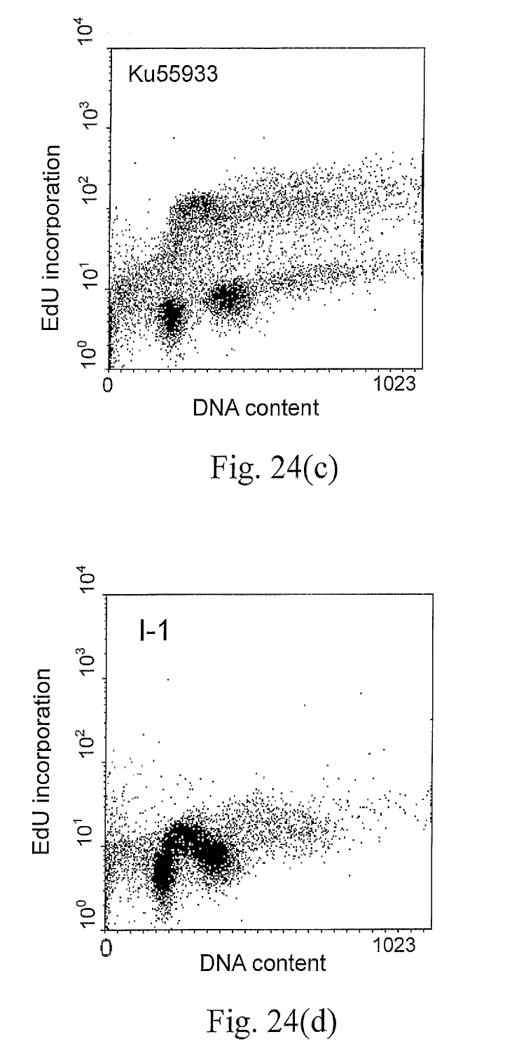

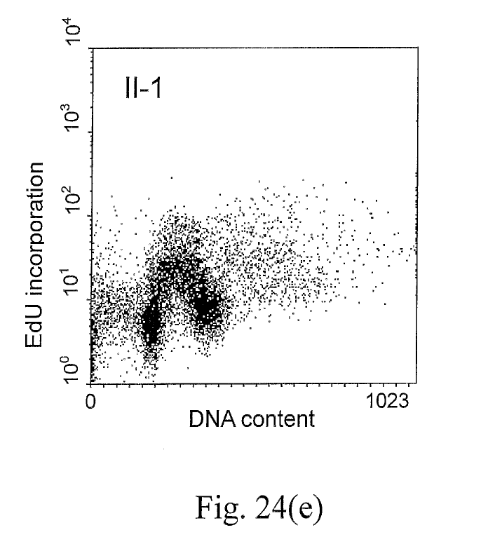

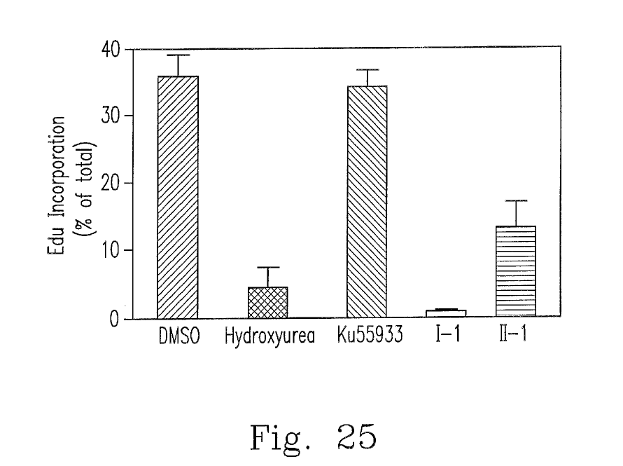

Through EdU (5-ethynyl-2′-deoxyuridine) incorporation to measurement the capacity of DNA replication, 4 μM protoapigenone (I-1) (FIGS. 24(d)) and 0.4 μM compound II-1 (FIG. 24(e)) but not 10 μM ATM inhibitor KU55933 (FIG. 24(C)) showed efficiently reduce the percentage of incorporation in compared with control group (FIG. 24(a)), indicating that DNA replication is inhibited (FIG. 25); these events are similar to the effect of a DNA replication blocker Hydroxyurea (HU) (FIG. 24(b)) and visualized that benzopyran-4-one derivatives with the ability to inhibit the DNA replication.

TABLE 4

| cell cycle | | | G1 | S | G2M |
|---|---|---|---|---|---|
| unsynchronized cells | | | 42.51% | 30.26% | 24.75% |
| initiation (0 hrs) | | Control | 70.95% | 24.00% | 1.29% |
| release | 6 hrs | Control | 18.29% | 75.63% | 0.63% |
| synchronizeation | | I-1 | 25.45% | 69.13% | 0.41% |
| | | II-1 | 21.72% | 72.01% | 4.59% |
| | 9 hrs | Control | 20.94% | 25.53% | 53.54% |
| | | I-1 | 21.41% | 60.89% | 16.46% |
| | | II-1 | 15.50% | 63.06% | 20.45% |

Materials and Methods

Antibodies

Primary antibodies of Chk1 (sc-8408), Chk2 (sc-17747), FANCD2 (SC-20022), phospho-ATM Ser1981 (sc-47739), and Myc (sc-40) were purchased from Santa Cruz. Phospho-histone H3 Ser10 (06-570) and H2AX Ser139 (05-636) antibodies were purchased from Millipore. Claspin (2880) and phospho-Chk1 Ser345 (2348), phospho-Chk2 Thr68 (2661), phospho-P53 Ser15 (9286), P38 MAPK Thr180/Tyr182 (9216), and MAPKAPK2 Thr334 (3007) were purchased from Cell Signaling. Actin (A2066), flag (F 1804), and hemagglutinin (H9658) antibodies were purchased from Sigma-Aldrich. ATR (A300-137A), ATRIP (A300-095A), and TopBP1 (A300-111A) antibodies were purchased from Bethyl; and anti-ATM (GTX70103) antibodies were purchased from Gene Tex.

Cell Culture and Treatment

MDA-MB-231 (breast adenocarcinoma; ATCC HTB-26, BCRC 60425) and A549 (lung adenocarcinoma; ATCC CCL-185, BCRC 60074) human cell lines were purchased from Bioresource Collection and Research Center (BCRC, Hsinchu, Taiwan), and were authenticated by American Type Culture Collection (ATCC, Manassas, Va.). U2OS (osteosarcoma), HeLa (cervical adenocarcinoma), and HEK 293T (embryonic kidney cells) human cell lines were provided by Dr. Sheau-Yann Shieh (Institute of Molecular Biology, Academia Sinica, Taipei, Taiwan). Cells were maintained in Dulbecco's modified Eagle's medium (DMEM, Sigma-Aldrich) supplemented with 10% fetal bovine serum (FBS) (Gibco). For DDR induction, freshly diluted H₂O₂ (Merck) was added to the culture medium 1 h before the cells were harvested. For UV irradiation treatment, the cells were irradiated for 10 J/m² by a cross-linker (UVP) 1 h prior to analysis. Protoapigenone (I-1) and compound II-1 were isolated and synthesized as described previously (15-17).

In Vitro Chemosensitization Assay

To evaluate in vitro chemosensitization, cells were seeded in 6-well plates 1 d before the experiment at a density of 100-400 cells/well. The drugs were incubated with the cells for 6 h, after which the medium was replaced with fresh drug-free FBS-containing medium. The colonies became visible and were counted 7-10 d later using 0.1% crystal violet staining following image capture by a CCD camera (LAS-4000 mini; Fujifilm).

Flow Cytometry

To evaluate the effect of DNA damage checkpoint activation on cell cycle distribution, the cells were harvested at indicated time points and fixed with methanol for at least 2 h. The DNA was then stained with a solution containing propidium iodide (PI) and RNase A (Sigma-Aldrich). Fluorescently labeled cells were subsequently analyzed by the flow cytometer (LSR II; BD Biosciences) with a suitable selection of excitation and emission wavelengths. The percentages of different fluorescent cell populations were analyzed using WinMDI Ver. 2.9 (The Scripps Research Institute).

DNA replication was measured using a Click-it EdU assay kit, which is based on incorporation of the thymidine analogue 5-ethynyl-2′-deoxyuridine (EdU) into DNA during replication (Invitrogen). Then, 10 μM EdU was added to the cell culture medium 30 min before the cells were harvested and fixed in 4% paraformaldehyde. After cycloaddition, EdU was detected with Alexa Fluor 647 using click reaction catalyzed by Cu (II), and the DNA content was stained by CellCycle 405-blue. To assay the mitotic entry, cells were treated with the indicated drugs and trapped in 70 nM nocodazole for 16 h, and antibodies against phospho-histone H3 Ser10 and PI were used to stain the mitotic cells and DNA content, respectively. An FITC Annexin V apoptosis detection kit was used to characterize the phenotype of cell death based on PI and Annexin V double staining (BD Pharmingen, San Diego, Calif.). Fluorescence-labeled cells were subsequently analyzed by the BD LSR II flow cytometer with a suitable selection of excitation and emission wavelengths. The percentages of different fluorescent cells were analyzed using WinMDI Ver. 2.9.

In Vitro Chromosome Aberration Test

In brief, 5×10⁵ Chinese Hamster Ovary (CHO) cells were seeded in 60-mm dishes 1 d before the experiment. Protoapigenone (I-1)-induced structural chromosomal changes after 20 h were compared with that of the cells cultured in 2 μM mitomycin C. At 18 h after Protoapigenone (I-1), 0.1 μg/mL colchicine was added for 2 h, and metaphase cells were collected by shaking them off the dishes. Mitotic cells were treated with 0.5% KCl for 10 min and fixed with a 3:1 mixture of methanol: glacial acetic acid. The cells were then spread on slides for chromosome staining with 5% Giemsa solution. It is then analyzed the chromosome structure of 200 well-spread metaphase cells (100 metaphase cells/experiment) under a 100× oil immersion objective.

Plasmids and siRNAs

The plasmids ATR, ATRIP, and claspin were kindly provided by Dr. X. Wu (The Scripps Research Institute, La Jolla, Calif.), and TopBP1 was provided by Dr. J. Chen (University of Texas MD Anderson Cancer Center, Houston, Tex.). The siRNA sequences of the target ATM (5′-AAGCGCCTGATTCGAGATCCT-3′), ATR (5′-CCTCCGTGATGTTGCTTGATT-3′), DNA-PKcs (5′-GATCGCACCTTACTCTGTTGA-3′), and the random sequence that served as the control (5′-AAGTCAATATGCGACTGATGG-3′) were synthesized by Sigma-Proligo (23,24). All transfections in HEK293T cells were performed by the calcium phosphate precipitation method.

Western Immunoblotting.

Cell lysate preparations, gel electrophoresis, and immunoblotting were performed as previously described (23). The binding of primary antibodies were detected by horseradish peroxidase-coupled secondary antibodies (Jackson ImmunoResearch) followed by enhanced chemiluminescence (ECL)(Millipore). The images of non-saturated bands were captured using a luminescent image analyzer (LAS-4000 mini; Fujifilm). The antibodies used in this study are listed in supplementary materials.

DNA Homologous Recombination Repair Assay.

DNA constructs of the recombination substrate pHPRT-DRGFP, in which the I-SceI site lies within 1 copy of 2 mutated tandem repeated GFP genes, and the I-SceI endonuclease expression vector pCBASceI, were originally constructed by Dr. M. Jasin (25). In brief, it is generated a stable pHPRT-DRGFP construct in HeLa cells, and evaluated the chromosomal breaks generated by I-SceI endonuclease expression. Six hours after pCBASceI was delivered into the cells, complete medium with or without Protoapigenone (I-1) or compound II-1 was replaced onto the cells. Forty-eight hours after delivery, the efficiency of chromosomal HRR was obtained as the percentage of GFP-positive cells, which was assessed by flow cytometry.

Human Xenograft Tumors in Nude Mice.

Human breast cancer MAD-MB-231 cells were harvested from the culture, resuspended in medium without serum at 1×10⁸ cells/mL, and 0.1 mL of this suspension was subcutaneously injected into the right flank of female nude mice (BALB/cAnN-Foxn1nu/Crl Narl; purchased from the National Science Council Animal Center, Taiwan). Tumor-injected mice that successfully developed tumors that grew to approximately 50-100 mm³ in volume were randomly sorted into groups and used for the experiments. Control vehicle or 2 mg/kg of cisplatin with or without 0.2 mg/kg of compound II-1 was administered intraperitoneally every 4 d throughout the experiment.

### Example 1

### Preparation of the Composition in Tablet

Tablets are prepared using standard mixing and formation techniques as described in U.S. Pat. No. 5,358,941, to Bechard et al., issued Oct. 25, 1994, which is incorporated by reference herein in its entirety.

| | |
|---|---|
| Protoapigenone (I-1) | 100 mg |
| Lactose | qs |
| Corn starch | qs |

### EMBODIMENTS

### Embodiment 1

A inhibiting of DNA Damage Response composition including a benzopyran-4-one derivatives compound presented by formula I:

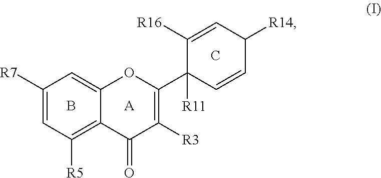

wherein: R₃, R₅, R₇, R₁₁, R₁₄ and R₁₆ are selected independently from a group consisting of a hydrogen, a hydroxyl group, a methoxyl group and a oxygen atom contain a double bond.

### Embodiment 2

A inhibiting of DNA Damage Response composition including a benzopyran-4-one derivatives compound presented by formula II:

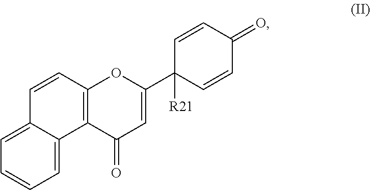

wherein: R₂₁ is selected independently from a group consisting of a hydrogen, a hydroxyl group and a methoxyl group.

### Embodiment 3

A medical effect for inhibited ATR-mediated DNA damage checkpoint composition including an effective amount of being one compound selected from a group consisting of benzopyran-4-one derivatives represented by formula I and formula II.

### Embodiment 4

A sensitizing effect for chemotherapeutic treatment composition including an effective amount of a compound selected from a group consisting of benzopyran-4-one derivatives represented by formula I and formula II.

### Embodiment 5

A assay composition for providing assay for the state of DNA DDR signaling cascade, including an effective amount of a compound selected from a group consisting of benzopyran-4-one derivatives represented by formula I and formula II.

### Embodiment 6

A pharmaceutical as above embodiments, therewith in a subject in need thereof including co-administration a compound of benzopyran-4-one derivatives; and at least one chemotherapeutic drugs against a cancer disease in need thereof.

### Embodiment 7

A pharmaceutical as above embodiments, therewith the chemotherapeutic drugs includes one selected from an alkylating agent, an antimetabolic agents, an antibiotic anti-cancer agents, a Topoisomerase I, a Topoisomerase II, an anti-mitosis agents and a combination thereof.

### Embodiment 8

A pharmaceutical as above embodiments, therewith the alkylating agent is one selected from Nitrogen mustards (eg. Melphalan, mechlorethamine, Chlorambucil, Ifosfamide, Cyclophosphamide, Estramustine and phenoxybenzamine); or Aziridines (eg. Thiotepa, Carboquone); or Nitrosoureas (eg. Carmustine, Semustine, Iomustine, Nimustine, Streptozocin, Ranimustine and Lomustine); or Procarbazine and triazenes (eg. Dacarbazine, Temozolomide and Procarbazine); or Alkyl sulfonate (eg. Busulfan); or Platinum coordination complex (eg. Cisplatin, Carboplatin, Nedaplatin, Iproplatin and Oxaliplatin); and a combination thereof.

### Embodiment 9

A pharmaceutical as above embodiments, therewith the antimetabolic agent is one selected from Thymidylate synthase inhibitor (eg. Aminopterin, Methotrexate, Tegafur, Piritrexin, Trimetrexate, Floxuridine, Raltitrexed, Pemetrexed, Fluorouracil, Doxifluridine and Capecitabine); or Amidophosphoribosyl transferase inhibitors (eg. Mercaptopurine, Thioguanine and Thionosine); or DNA chain elongation inhibitors (eg. Cytarabine, Ancitabine, Gemcitabine, Fludarabine, Cladribine, Clofarabine, Azaserine, Azacitidine, Pentostatin, Hydroxyurea); and a combination thereof.

### Embodiment 10

A pharmaceutical as above embodiments, therewith the antibiotic anti-cancer agent is one selected from free radical agents (eg. Bleomycin and Actinomycin D); or Topoisomerase II inhibitors (eg. Daunorubicin, Doxorubicin, Idarubicin, Epirubicin, valrubicin, Pirarubicin, Aclarubicin, Mitoxantrone and Piroxanthrone); or other therapies or anticancer agents (eg. Menogaril, Plicamycin, Acivicin, Anthramycin, Pentostatin, Calicheamicin and Peplomycin) and a combination thereof.

### Embodiment 11

A pharmaceutical as above embodiments, therewith the Topoisomerase inhibitor is one selected from Topoisomerase I inhibitors (eg. Camptothecin, Irinotecan, Topotecan); or Topoisomerase II (eg. Podophyllin, Podophyllotoxin, Etoposide, Teniposide) and a combination thereof.

### Embodiment 12

A pharmaceutical as above embodiments, therewith the anti-mitosis agent is one selected from Paclitaxel and Docetaxel; or anti-microtubule agents (eg. Colchicine, Vinblastine, Vincristine, Vindesine and Vinorelbine) and a combination thereof.

### Embodiment 13

A method for sensitizing cells to DNA damaging agents, including steps of providing an effective amount of a benzopyran-4-one derivative; and administering the effective amount of the benzopyran-4-one derivative to a subject in need thereof.

### Embodiment 14

A method for inhibiting a DNA damage response (DDR), including steps of providing an effective amount of a benzopyran-4-one derivative; and administering the effective amount of the benzopyran-4-one derivative to a subject in need thereof.

### Embodiment 15

A method as above embodiments, wherein the benzopyran-4-one derivative is represented by formula I:

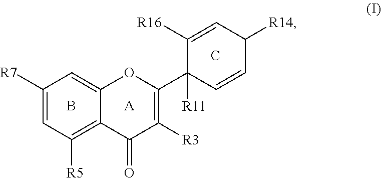

wherein each of R₃, R₅, R₇, R₁₁, R₁₄ and R₁₆ is one selected from a group consisting of a hydrogen, a hydroxyl group, a methoxyl group and an oxygen atom containing a double bond.

### Embodiment 16

A method as above embodiments, wherein the benzopyran-4-one derivative is represented by formula II:

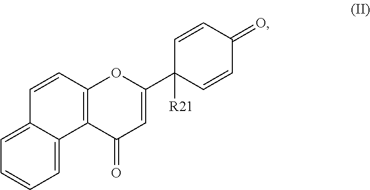

wherein R₂₁ is one selected from a group consisting of a hydrogen, a hydroxyl group and a methoxyl group.

### Embodiment 17

A method as above embodiments, wherein the administering step further includes a step of co-administering the benzopyran-4-one derivative and at least one of chemotherapeutic drug against a cancer disease to the subject in need thereof.

### Embodiment 18

A method as above embodiments, wherein the chemotherapeutic drugs include one selected from a group consisting of an alkylating agent, an antimetabolic agent, an antibiotic anti-cancer agent, a Topoisomerase I, a Topoisomerase II, an anti-mitosis agent and a combination thereof.

### Embodiment 19

A method for inhibiting an ATR-mediated DNA damage checkpoint, including a step of administering to a subject in need thereof an effective amount of a compound being one of formula I and formula II,

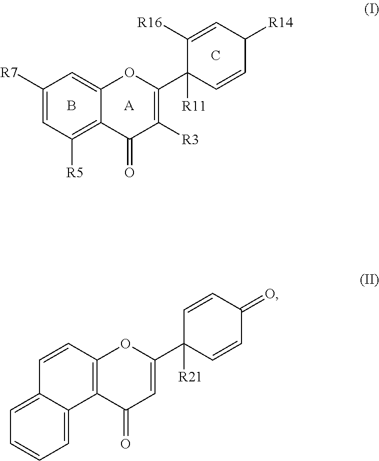

wherein each of R₃, R₅, R₇, R₁₁, R₁₄, R₁₆ is one selected from a group consisting of a hydrogen, a hydroxyl group, a methoxyl group and an oxygen atom containing a double bond; wherein R₂₁ is one selected from a group consisting of a hydrogen, a hydroxyl group and a methoxyl group.

### REFERENCES

Andreassen P R, et al. ATR couples FANCD2 monoubiquitination to the DNA-damage response. Genes Dev 2004; 18(16):1958-63.
Chen H M, et al. A novel synthetic protoapigenone analogue, WYC02-9, induces DNA damage and apoptosis in DU145 prostate cancer cells through generation of reactive oxygen species. Free Radic Biol Med 2011; 50(9):1151-62.
Chen W Y, et al. Protoapigenone, a natural derivative of apigenin, induces mitogen-activated protein kinase-dependent apoptosis in human breast cancer cells associated with induction of oxidative stress and inhibition of glutathione S-transferase pi. Invest New Drugs 2011; 29(6):1347-59.
Chirnomas D, et al. Chemosensitization to cisplatin by inhibitors of the Fanconi anemia/BRCA pathway. Mol Cancer Ther 2006; 5(4):952-61.
Chiu C C, et al. Fern plant-derived protoapigenone leads to DNA damage, apoptosis, and G(2)/m arrest in lung cancer cell line H1299. DNA Cell Biol 2009; 28(10):501-6.
Lopez-Contreras A J, et al. The ATR barrier to replication-born DNA damage. DNA Repair (Amst) 2010; 9(12):1249-55.
Nghiem P, et al. ATR inhibition selectively sensitizes G1 checkpoint-deficient cells to lethal premature chromatin condensation. Proc Natl Acad Sci USA 2001; 98(16):9092-7.
Sorensen C S, et al. The cell-cycle checkpoint kinase Chk1 is required for mammalian homologous recombination repair. Nat Cell Biol 2005; 7(2): 195-201.
Wang H C, et al. Inhibition of ATR-dependent signaling by protoapigenone and its derivative sensitize cancer cells to interstrand cross-link-generating agents in vitro and in vivo. Mol Cancer Ther molcanther. Apr. 24, 2012; 1443

What is claimed is:

1. A method for inhibiting a DNA damage response (DDR), comprising steps of: providing an effective amount of a benzopyran-4-one derivative; and administering the effective amount of the benzopyran-4-one derivative to a subject in need thereof.

2. A method as claimed in claim 1, wherein the benzopyran-4-one derivative is represented by formula I: 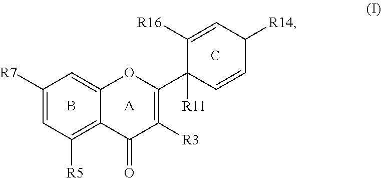
wherein each of R₃, R₅, R₇, R₁₁, R₁₄ and R₁₆ is one selected from a group consisting of a hydrogen, a hydroxyl group, a methoxyl group and an oxygen atom containing a double bond.

3. A method as claimed in claim 1, wherein the benzopyran-4-one derivative is represented by formula II: 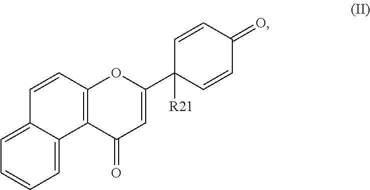
wherein R₂₁ is one selected from a group consisting of a hydrogen, a hydroxy and a methoxyl group.

4. A method as claimed in claim 1, wherein the administering step further comprises a step of co-administering the benzopyran-4-one derivative and at least one of chemotherapeutic drug against a cancer disease to the subject in need thereof.

5. A method as claimed in claim 4, wherein the chemotherapeutic drugs include one selected from a group consisting of an alkylating agent, an antimetabolic agent, an antibiotic anti-cancer agent, a Topoisomerase I, a Topoisomerase II, an anti-mitosis agent and a combination thereof.

6. A method for inhibiting an ATR-mediated DNA damage checkpoint, comprising a step of:
providing an effective amount of a benzopyran-4-one derivative; and
administering the effective amount of the benzopyran-4-one derivative to a subject in need thereof.

7. A method as claimed in claim 6, wherein the administering step further comprises a step of co-administering the compound and at least one chemotherapeutic drugs against a cancer disease to the subject in need thereof.

8. A method as claimed in claim 7, wherein the chemotherapeutic drugs include one selected from a group consisting of an alkylating agent, an antimetabolic agent, an antibiotic anti-cancer agent, a Topoisomerase I, a Topoisomerase inhibitors II, an anti-mitosis agent, a DNA-damaging agent and a combination thereof.

9. An assaying method for a state of DNA DDR kinase signaling cascades, comprising the steps of:
providing a reaction site thereof;
adding to the reaction site an effective amount of a benzopyran-4-one derivative.

10. A method as claimed in claim 9, wherein each the DNA DDR kinase signaling cascades includes at least one cell marker, and each the cell marker is one of an altered expression cell marker and an altered activity cell marker, and is one selected from a group consisting of ATM, CHK1, CHK2, p53, AMPK, mTORC1, MRE 11, MAPK, MAPKAPK 2, RAD50, NBS 1, 53BP1, MDC 1, H2AX and a combination thereof.

11. A method as claimed in claim 9, wherein each the DNA DDR kinase signaling cascades includes at least one cell marker, and each the cell marker is one of an altered expression cell marker and an altered activity cell marker, and is one selected from a group consisting of K-Ras, N-Ras, H-Ras, Raf, Myc, Mos, E2F, Cdc25A, CDC4, CDK2, Cyclin E, Cyclin A and Rb, and a combination thereof.

12. A method as claimed in claim 9, wherein the adding step further comprises a step of co-adding the benzopyran-4-one derivative and at least one of chemotherapeutic drugs against a cancer disease to the reaction site.

13. A method as claimed in claim 12, wherein the chemotherapeutic drugs include one selected from a group consisting of an alkylating agent, an antimetabolic agent, an antibiotic anti-cancer agent, a Topoisomerase I, a Topoisomerase inhibitors II, an anti-mitosis agent and a combination thereof.

14. A method as claimed in claim 13, wherein the alkylating agent is one selected from a group consisting of a Nitrogen mustard, an Aziridine, a Nitrosourea, a Procarbazine, a triazene, an Alkyl sulfonate, a Platinum coordination complex and a combination thereof.

15. A method as claimed in claim 13, wherein the antimetabolic agent is one selected from a group consisting of a Thymidylate synthase inhibitor, an Amidophosphoribosyl transferase inhibitor, a DNA chain elongation inhibitor and a combination thereof.

16. A method as claimed in claim 13, wherein the antibiotic anti-cancer agent is one selected from a group consisting of a free radical agent, a Topoisomerase II inhibitor, an anticancer agent and a combination thereof.

17. A method as claimed in claim 13, wherein the Topoisomerase inhibitor is one selected from a group consisting of a Topoisomerase I inhibitor, a Topoisomerase II and a combination thereof.

18. A method as claimed in claim 13, wherein the anti-mitosis agent is one selected from a group consisting of a Paclitaxel, a Docetaxel, an anti-microtubule agent, and a combination thereof.


SEQUENCE LISTING (4 sequences)

SEQ ID NO: 1 - DNA, 21 bp, Homo sapiens
  misc_feature (1)..(21): Human ATM specific siRNA sequence
aagcgcctgattcgagatcct

SEQ ID NO: 2 - DNA, 21 bp, Homo sapiens
  misc_feature (1)..(21): Human ATR specific siRNA sequence
cctccgtgatgttgcttgatt

SEQ ID NO: 3 - DNA, 21 bp, Homo sapiens
  misc_feature (1)..(21): Human DNA-PKcs specific siRNA sequence
gatcgcaccttactctgttga

SEQ ID NO: 4 - DNA, 21 bp, Homo sapiens
  misc_feature (1)..(21): Random siRNA sequence
aagtcaatatgcgactgatgg